(19) United States
(12) Patent Application Publication
(10) Pub. No.: US 2024/0301514 A1
(43) Pub. Date: Sep. 12, 2024

(54) ORAL SWAB-BASED TEST FOR THE DETECTION OF VARIOUS DISEASE STATES IN DOMESTIC CATS

(71) Applicant: BASEPAWS, Torrance, CA (US)

(72) Inventors: Damian Kao, Playa Vista, CA (US); Yuliana Mihaylova, Playa Vista, CA (US)

(21) Appl. No.: 18/578,289

(22) PCT Filed: Jul. 14, 2022
(86) PCT No.: PCT/US22/73735

§ 371 Date: Jan. 10, 2024

Related U.S. Application Data
(60) Provisional application No. 63/221,558, filed on Jul. 14, 2021.
(60) Provisional application No. 63/221,559, filed on Jul. 14, 2021.

(51) Int. Cl. C12Q 1/689; G16H 50/20; G16H 50/30

(52) U.S. Cl. CPC C12Q 1/689; G16H 50/20; G16H 50/30; C12Q 2600/158

(57) ABSTRACT

Systems and methods for screening for, diagnosing, indicating, treating, and identifying renal/urinary, inflammatory, and/or endocrine disease states in domestic cats. Systems and methods for screening for, diagnosing, indicating, treating, and identifying renal/urinary, inflammatory, and/or endocrine disease states in domestic cats. Systems and methods for screening for, diagnosing, indicating, treating, and identifying renal/urinary, inflammatory, and/or endocrine disease states in domestic cats.

### CROSS-REFERENCE TO RELATED APPLICATIONS

This application is a nationalization of PCT/US22/73735, filed Jul. 14, 2022, which claims the benefit of and priority to U.S. Provisional Application No. 63/221,558, filed Jul. 14, 2021 and U.S. Provisional Application No. 63/221,559, filed Jul. 14, 2021. The entire contents of each of the foregoing are incorporated herein by specific reference.

### BACKGROUND

### Technical Field

This disclosure relates to systems and methods for screening for, detecting, diagnosing, and identifying renal and/or urinary, inflammatory, or endocrine disease states in domestic cats.

### Related Technology

Nutritional and environmental factors, as well as disease states, play an important role in the dynamic microbial composition of the mouth (i.e., the oral microbiome). With the mouth being the first line of defense from a constant barrage of foreign microbes, its microbiome has evolved to be competitive and territorial. The state of the oral microbiome has shown strong correlations with both dental and systemic health. For example, existing studies in humans have shown a connection or correlation between a human's oral microbiome and the presence of chronic kidney disease (CKD). Domestic animals, such as cats, are also at risk for developing renal and/or urinary diseases, such as CKD.

Many cats do not receive routine veterinary care, meaning that early signs of renal and/or urinary diseases can often be missed. Early signs or symptoms of some diseases, such as CKD, typically do not present with clinical signs and, therefore, may go unnoticed and undiagnosed. Further, early symptoms of some renal/urinary diseases, such as CKD, are non-specific (lethargy, weakness, vomiting, etc.), meaning pet owners might not identify the symptoms as indicative of a condition needing veterinarian assistance and/or diagnosis.

To compound this problem, urinalysis, which is critical for diagnosing renal and/or urinary conditions, is rarely a part of routine veterinary visits due to the difficulty of obtaining a feline urine sample. Obtaining such a sample requires performing cystocentesis, which is a procedure where a sterile needle and syringe are used to collect urine from the bladder. The reason why cystocentesis does not always work is because the cat may not have a full bladder during the veterinary visit. Ensuring that early signs of developing renal and/or urinary disease are not missed during an examination requires for the animal to have routine blood testing and urinalysis performed. Due to the already mentioned difficulties with urinalysis and the cost of prophylactic veterinary care, few cats undergo these procedures on a routine basis. As a result, most cases of chronic kidney disease (CKD) are detected at their late stages when treatment options are limited and disease progression is rapid. Urinary crystals and stones also often go undiagnosed until the cat is in severe pain and may have a blockage of the urethra before they see a veterinarian.

Current tools for early diagnosis or detection of renal/urinary diseases require, or rely on, a pet owner keeping up with their 6 to 12-month routine vet visits. As already mentioned, many pet owners do not keep up with their routine vet visits. Additionally, current tools require veterinarians to perform serum and urine diagnostic screenings during those routine visits. This is not currently a common practice, unless a cat is older than about 6-8 years or the cat is already symptomatic of CKD. Other diseases, such as inflammatory bowel disease (IBD) and diabetes mellitus (DM), are also problematic in cats. Such disease may also be missed by a veterinarian and not diagnosed until the disease has progressed to a later stage and treatment options are not plentiful.

Accordingly, there is a need for robust and accurate, yet safe, painless and affordable means that can be used on a recurring basis for detecting various feline diseases including renal and/or urinary, inflammatory, and/or endocrine diseases.

### SUMMARY

Embodiments of the present disclosure include systems and methods for screening for, detecting, diagnosing, treating, and/or identifying one or more disease states in cats. For example, embodiments of the present disclosure include system and methods for screening for, detecting, diagnosing, and/or identifying renal diseases, urinary diseases, inflammatory diseases and/or endocrine diseases. Using such tools to guide and complement veterinary health assessment can significantly improve renal and/or urinary health outcomes. Embodiments of the present disclosure lead to earlier detection of deteriorating kidney or urinary functions and earlier implementation of treatment compared to relying on veterinary visits alone. Embodiments of the disclosed subject matter describe a method for interrogating the oral microbiome of a cat. The disclosed methods interrogate the oral microbiome to detect microbe compositional abundance trends that may be associated with renal and/or urinary diseases in cats. Detecting, identifying and/or quantifying microbial compositional abundance trends enables a practitioner to screen for and/or indicate whether a cat has a particular renal and/or urinary disease state. Detecting and identifying renal and/or urinary disease states enables the practitioner and/or the cat's owner to treat and/or prevent the renal/urinary disease state.

In some embodiments, a method is disclosed for detecting and/or indicating renal/urinary diseases in cats. The method may include receiving an oral swab sample taken from a cat; manipulating the sample, such as heat treatment of the oral sample; and extracting microbial deoxyribonucleic acids (DNA) from the heat-treated sample. The method may additionally include sequencing the microbial DNA to identify which specific one or more microbes are present in the oral sample (and in what relative proportions), wherein identifying the specific one or more microbes enables generation of an oral microbial profile for the cat. The method may additionally include comparing the oral microbial profile for the cat against a reference database including defined microbial profiles, wherein the database identifies correlations between (i) profiles that include one or more microbes, and (ii) corresponding renal/urinary diseases; and based on a result of comparing the oral microbial profile against the database of defined microbial profiles, generating a risk score indicating a likelihood that the cat has a specific renal/urinary disease.

The method may further include treating the specific renal/urinary disease and/or administering a therapeutic treatment. In some embodiments, the therapeutic treatment may include administering a therapeutic compound, such as a compound designed to inhibit or encourage growth of a specific one or more microbes present in the oral microbiome of the mammal. In some embodiments, the therapeutic compound includes a pre-biotic, a post-biotic, a pro-biotic, a medicament or a combination thereof. In some embodiments, the therapeutic compound includes a phosphate binder, an antibiotic, a compound to control hypertension and/or blood pressure of the cat, and erythropoietin, among other therapeutic compounds. In some embodiments, the therapeutic treatment may include brushing the mammal's teeth with a topical treatment.

In some embodiments, the therapeutic treatment may include a dietary regimen designed to address and/or alleviate the renal/urinary disease state. For example, therapeutic diets that are restricted in protein, phosphorus and sodium content, and high in water-soluble vitamins, fiber, and antioxidant concentrations, may prolong life and improve quality of life in cats with CKD. In some embodiments, the dietary regimen may include switching to a wet food to help maintain proper hydration of the cat. In some embodiments, the dietary regimen may be designed to treat or manage IBD and/or DM. The therapeutic treatment may include potassium supplementation, and other nutritional or vitamin supplementation.

In some embodiments, a method for indicating a disease (e.g., a renal/urinary disease, IBD and/or diabetes) in cats includes receiving an oral swab sample taken from a cat and performing heat treatment on the oral sample. The method may also include performing magnetic beads-based deoxyribonucleic acid (DNA) extraction on the heat treated oral sample to extract microbial DNA that is present in the oral swab sample and sequencing the microbial DNA to identify which specific one or more microbes are present in the oral sample (and in what compositional abundance), wherein identifying the specific one or more microbe(s) enables generation of an oral microbial profile for the cat. The method may additionally include comparing the oral microbial profile for the cat against a database of defined microbial profiles, wherein the database identifies correlations between (i) profiles that include one or more microbes (and their compositional abundance), and (ii) corresponding diseases (e.g., renal/urinary disease, IBD and/or diabetes); and based on a result of comparing the oral microbial profile against the database of defined microbial profiles, generating a risk score indicating a likelihood that the cat has a disease. The method may include, in response to generating the risk score and identifying the specific disease (e.g., renal/urinary disease, IBD and/or diabetes), administering a therapeutic treatment designed to treat the specific disease, recommending veterinary attention or follow-up examination, and/or recommending at-home care for specific diseases (e.g., renal/urinary disease, IBD and/or diabetes).

Also disclosed are computer systems. In some embodiments, a computer system is configured to indicate one or more diseases (e.g., a renal/urinary disease, IBD and/or diabetes) in cats and includes one or more processors and one or more computer-readable hardware storage devices that store instructions executable by the one or more processors. The instructions may configure the computer system to receive sequenced microbial DNA data from an oral swab sample taken from a cat; map the sequenced microbial DNA to identify which specific one or more microbial species are present in the oral sample, wherein identifying the specific one or more microbial species results in generation of an oral microbial profile for the cat; calculate a relative abundance of different microbial species to further build the oral microbial profile; compare the oral microbial profile against a database of defined microbial profiles, wherein the database identifies correlations between (i) profiles that include one or more microbial species and their relative abundance(s), and (ii) corresponding one or more diseases (e.g., renal/urinary diseases, IBD, and/or diabetes); and based on a result of comparing the oral microbial profile against the database of defined microbial profiles, generate a risk score indicating a likelihood that the cat has a specific disease (e.g., a renal/urinary disease, IBD and/or diabetes). In response to generating the risk score, the instructions may further configure the computer system to generate a report outlining and/or presenting the risk score and prescribing a therapeutic treatment and/or at-home treatment protocol suitable for addressing (e.g., treating, arresting and/or preventing) the specific disease. The therapeutic treatment protocol may be influenced by the severity of the disease state, which is indicated by or correlated to the risk score.

For example, in some embodiments, the risk score may incorporate or correlate to approximately three (3) risk assessment categories based on the risk/probability score generated: a 0.0-0.33 bracket is classified as ‘low risk’ of having a renal or urinary condition; >0.33-0.66 is classified as ‘medium risk’ for having a renal or urinary condition; and >0.66-1.0 is classified as ‘high risk’ for having a renal or urinary condition. For example, a risk score of 0.34 would meet the threshold for categorizing a cat as being at medium risk for having a renal or urinary condition. The granularity of the risk score and/or the number of categories may change as more data is added to the systems and methods.

In some embodiments, the therapeutic treatment or at-home care protocol can alter the composition of the oral microbiome of the cat directly or as a byproduct of the treatment of a specific condition (e.g., a renal/urinary disease, IBD and/or diabetes). In some embodiments, altering the composition of the cat's oral microbiome treats and/or addresses the specific disease or condition. In some embodiments, the therapeutic treatment repairs the cat's oral microbiome. In some embodiments, repairing the cat's oral microbiome brings the cat's oral microbiome more in line with the oral microbiome (or defined oral microbial profile) of a healthy cat-both in terms of the specific one or more microbial species present and their relative abundance. In some embodiments, the therapeutic treatment or at-home care protocol is designed to maintain the composition of the oral microbiome of the cat. In some embodiments, the therapeutic treatment protocol is designed to stimulate a metabolic output of the cat's oral microbiome. Stimulating a metabolic output of the cat's oral microbiome may include using known enzymatic pathway analysis tools to provide an additional dimension to the existing microbial composition data to further characterize disease signatures and improve predictive disease models.

Illustrative embodiments and non-limiting examples of the present disclosure include:
Example 1. A method for screening for, detecting, and/or preventing one or more diseases in domestic cats, the method comprising:
   obtaining an oral microbial profile for a cat, the oral microbial profile comprising one or more microbial species present in an oral sample of the cat and a quantity or abundance of the one or more microbial species in the oral sample;
   comparing the oral microbial profile to information in a database that identifies weighted correlations between:
   (i) occurrence and/or prevalence of one or more diseases (e.g., a renal/urinary disease, IBD and/or diabetes) in cats; and
   (ii) presence and/or abundance of various microbial species in the oral microbiome of cats, wherein the various microbial species comprise the one or more microbial species in the oral sample;
   generating a risk score indicating a likelihood that the cat has the one or more renal/urinary diseases based on one or more matches between the oral microbial profile and the information in the database; and
   categorizing the cat as developing the one or more diseases (e.g., a renal/urinary disease, IBD and/or diabetes) when the risk score meets or exceeds a predetermined threshold and, optionally, prescribing a therapeutic treatment protocol suitable for treating, mitigating, or preventing the development, advancement, or recurrence of the one or more diseases when the risk score meets or exceeds a predetermined threshold.
Example 2. The method of Example 1 further comprising administering the therapeutic treatment protocol to the cat or confirming that the therapeutic treatment protocol has been administered to the cat, wherein the therapeutic treatment protocol is sufficient to alter the oral microbial profile of the cat.
Example 3. The method of Example 1, wherein obtaining the oral microbial profile for the cat comprises:
   obtaining nucleic acid sequence data corresponding to microbial nucleic acid obtained from the oral sample;
   analyzing the nucleic acid sequence data to identify the one or more microbial species present in the oral sample and, optionally, quantifying the one or more microbial species; and
   generating the oral microbial profile for the cat based on the identified and, optionally, quantified one or more microbial species.
Example 4. The method of Example 3, wherein obtaining the microbial nucleic acid sequence data comprises:
   sequencing microbial nucleic acid from the oral sample; and, optionally,
   isolating the microbial nucleic acid from the oral sample.
Example 5. The method of Example 4, wherein isolating the microbial nucleic acid from the oral sample comprises:
   performing heat treatment on the oral sample; and
   performing magnetic SPRI beads-based nucleic acid extraction on the heat-treated oral sample, with or without the addition of protein digesting reagents and detergents, to extract the microbial nucleic acid from the oral sample.
Example 6. The method of Example 3, wherein analyzing the microbial nucleic acid sequence data comprises one or more of:
   demultiplexing the nucleic acid sequence data;
   trimming the nucleic acid sequence data;
   mapping one or more unmapped reads onto a reference genome of the cat and/or onto existing microbial reference genomes;
   classifying one or more reads as feline from the nucleic acid sequence data after mapping;
   classifying one or more reads as microbial from the nucleic acid sequence data after mapping;
   quantifying the one or more microbial reads;
   transforming the quantified one or more microbial reads to account for sequence coverage biases using methods such as pairwise log ratio transformation; and
   comparing compositional abundance patterns in the transformed one or more microbial reads against compositional abundance patterns in transformed data in a reference database comprising samples from cats that do not suffer from renal/urinary diseases, as well as samples from cats that suffer from specific diseases (e.g., renal/urinary diseases, IBD and/or diabetes).
Example 7. The method of Example 1, wherein comparing the oral microbial profile for the cat to the information in the database comprises one or more of:
   calculating the abundance of the one or more microbial species in the oral sample;
   identifying the one or more microbial species in the oral sample; and
   comparing the abundance of the identified one or more microbial species in the oral sample to the presence and/or abundance of various microbial species in the oral microbiomes of cats contained in the database.
Example 8. The method of Example 1, wherein generating the risk score comprises one or more of:
   identifying one or more similarities between compositional abundance(s) of the one or more microbial species in the oral sample and compositional abundance(s) of various microbial species in the oral microbiomes of cats contained in the database;
   identifying one or more matches between the identities of the one or more microbial species in the oral sample and the presence of various microbial species in the oral microbiomes of cats contained in the database;
   quantifying the identified one or more similarities between the compositional abundance of the one or more microbial species in the oral sample and the compositional abundance of the one or more microbial species in the oral microbiomes of cats contained in the database; and
   identifying a presence of one or more predictive microbial species in the oral sample.
Example 9. The method of Example 1, wherein the one or more diseases is selected from the group consisting of IBD, DM, CKD, struvite urinary crystals or stones, urinary calcium oxalate crystals or stones, cystine urinary crystals or stones, or idiopathic cystitis.
Example 10. The method of Example 1 further comprising:
   generating a report presenting (i) the risk score, (ii) an indication of developing the one or more diseases (e.g., a renal/urinary disease, IBD and/or diabetes) when the risk score meets or exceeds the predetermined threshold, (iii) a timing recommendation, (iv) optionally, one or more at home practices to improve renal/urinary health, (v) optionally, one or more diagnostic steps to diagnose the one or more renal/urinary diseases when the risk score meets or exceeds the predetermined threshold, and (vi) optionally, a prescription for the therapeutic treatment protocol; and, optionally,
   communicating the generated report electronically to an owner of the cat and/or their veterinarian.
Example 11. The method of Example 1, wherein the therapeutic treatment protocol is sufficient to alter the oral microbial profile of the cat.
Example 12. A computer system configured to indicate or predict one or more disease states in cats, the computer system comprising:
   one or more processors; and
   one or more computer-readable hardware storage devices having stored thereon instructions that are executable by the one or more processors to configure the computer system to:
   receive microbial nucleic acid sequence data corresponding to microbial nucleic acid obtained from an oral sample taken from a cat;
   analyze the microbial nucleic acid sequence data to identify one or more microbial species present in the oral sample and quantify the one or more microbial species;
   generate an oral microbial profile for the cat based on the identified one or more microbial species and their respective abundances;
   compare the oral microbial profile to information in a database that identifies weighted correlations between:
   (i) occurrence and/or prevalence of one or more disease states in cats; and
   (ii) presence and/or abundance of various microbial species in oral microbiomes of cats, wherein the various microbial species comprise the one or more microbial species in the oral sample;
   identify one or more matches between the oral microbial profile and the information in the database;
   generate a risk score indicating a likelihood that the cat has the one or more renal/urinary diseases based on the one or more matches between the oral microbial profile and the information in the database; and, optionally,
   diagnose the cat as “developing” the one or more disease states when the risk score meets or exceeds a predetermined threshold,
   prescribe a therapeutic treatment protocol suitable for treating or preventing the one or more disease states when the risk score meets or exceeds the predetermined threshold,
   generate a report indicating (i) the risk score, (ii) an indication of developing the one or more disease states when the risk score meets or exceeds the predetermined threshold, (iii) a timing recommendation, (iv) optionally, one or more at home practices to improve health, (v) optionally, one or more diagnostic steps to diagnose the one or more disease states when the risk score meets or exceeds the predetermined threshold, and (vi) a prescription for the therapeutic treatment protocol, and/or
   communicate the generated report electronically to an owner of the cat and/or their veterinarian.
Example 13. The computer system of Example 12, wherein the instructions further configure the computer system to analyze metagenomic sequence data from the oral sample and map one or more unmapped sequence reads to a feline reference genome and/or map one or more sequence reads to microbial reference genomes and, optionally, classify the reads as microbial or feline.
Example 14. The computer system of Example 13, wherein the instructions further configure the computer system to identify at least one unmapped sequence read of the metagenomic sequence data and, optionally, classify the at least one unmapped read.
Example 15. The computer system of Example 13, wherein feline oral microbiome samples having fewer than 10,000 classified microbial reads or more than 500,000 classified microbial reads are excluded from the comparison of the oral microbial profile for the cat against a database of defined microbial profiles.
Example 16. The computer system of Example 12, wherein the instructions further configure the computer system to calculate an abundance of the one or more microbial species present in the oral sample.
Example 17. The computer system of Example 16, wherein the abundance of the specific one or more microbial species present in the oral sample correlates to whether the specific one or more microbial species is a predictive microbial species for the specific disease states.
Example 18. The computer system of Example 16, wherein the instructions further configure the computer system to perform a pairwise log ratio comparison of the microbial abundance of the cat's oral sample against the information in the database.
Example 19. The system of Example 18, wherein the specific one or more microbial species is a predictive microbial species when 50% or more of the maximum possible pairwise log ratio comparisons involving this microbe are significantly different when compared between a disease and a control cohort.
Example 20. A method for predicting the development of a disease state in a cat, the method comprising:
   obtaining an oral sample from a cat, the oral sample comprising one or more microbial species;
   isolating, from the oral sample, microbial nucleic acid of the one or more microbial species;
   obtaining microbial nucleic acid sequence data corresponding to the microbial nucleic acid;
   analyzing the microbial nucleic acid sequence data to identify the one or more microbial species present in the oral sample and, optionally, quantifying the one or more microbial species;
   generating an oral microbial profile for the cat based on the identified and, optionally, quantified one or more microbial species, the oral microbial profile comprising the one or more microbial species and, optionally, a quantity or relative abundance of the one or more microbial species in the oral sample;
   comparing the oral microbial profile to information in a database that identifies weighted correlations between:
   (i) occurrence and/or prevalence of one or more diseases in cats; and
   (ii) presence and/or abundance of various microbial species in oral microbiomes of cats, wherein the various microbial species comprise the one or more microbial species in the oral sample;
   generating a risk score indicating a likelihood of the cat developing the one or more diseases based on one or more matches between the oral microbial profile and the information in the database; and
   indicating the cat as developing the one or more diseases when the risk score meets or exceeds a predetermined threshold.
Example 21. A method for diagnosing a disease in a cat, the method comprising:
   obtaining an oral sample from a cat, the oral sample comprising one or more microbial species;
   isolating, from the oral sample, microbial nucleic acid of the one or more microbial species;
   obtaining microbial nucleic acid sequence data corresponding to the microbial nucleic acid;
   analyzing the microbial nucleic acid sequence data to identify the one or more microbial species present in the oral sample and, optionally, quantifying the one or more microbial species;
   generating an oral microbial profile for the cat based on the identified and, optionally, quantified one or more microbial species, the oral microbial profile comprising the one or more microbial species and, optionally, a quantity or relative abundance of the one or more microbial species in the oral sample;
   comparing the oral microbial profile to information in a database that identifies weighted correlations between:
   (i) occurrence and/or prevalence of one or more diseases in cats; and
   (ii) presence and/or abundance of various microbial species in oral microbiomes of cats, wherein the various microbial species comprise the one or more microbial species in the oral sample;
   generating a risk score indicating a likelihood of the cat developing the one or more diseases based on one or more matches between the oral microbial profile and the information in the database; and
   diagnosing the cat as developing the one or more diseases when the risk score meets or exceeds a predetermined threshold.
Example 22. The method of Example 23, wherein the one or more diseases are selected from the group consisting of inflammatory bowel disease, diabetes mellitus, chronic kidney disease, struvite urinary crystals or stones, urinary calcium oxalate crystals or stones, cystine urinary crystals or stones, or idiopathic cystitis
Example 23. A method for treating a renal and/or urinary, inflammatory, or endocrine disease in a cat, the method comprising:
   obtaining an oral sample from a cat, the oral sample comprising one or more microbial species;
   isolating, from the oral sample, microbial nucleic acid of the one or more microbial species;
   obtaining microbial nucleic acid sequence data corresponding to the microbial nucleic acid;
   analyzing the microbial nucleic acid sequence data to identify the one or more microbial species present in the oral sample and, optionally, quantifying the one or more microbial species;
   generating an oral microbial profile for the cat based on the identified and, optionally, quantified one or more microbial species, the oral microbial profile comprising the one or more microbial species and, optionally, a quantity or relative abundance of the one or more microbial species in the oral sample;
   comparing the oral microbial profile to information in a database that identifies weighted correlations between:
   (i) occurrence and/or prevalence of one or more renal and/or urinary, inflammatory, or endocrine diseases in cats; and
   (ii) presence and/or abundance of various microbial species in oral microbiomes of cats, wherein the various microbial species comprise the one or more microbial species in the oral sample;
   generating a risk score indicating a likelihood of the cat developing the one or more renal and/or urinary, inflammatory, or endocrine diseases based on one or more matches between the oral microbial profile and the information in the database;
   diagnosing the cat as developing the one or more renal and/or urinary, inflammatory, or endocrine diseases when the risk score meets or exceeds a predetermined threshold; and
   administering a therapeutic treatment, wherein the therapeutic treatment is sufficient to treat the one or more renal and/or urinary, inflammatory, or endocrine diseases.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Figures, like reference numerals may be utilized to designate corresponding or similar parts in the various Figures, and the various elements depicted are not necessarily drawn to scale, wherein:

FIG. 1A-1B illustrates a renal/urinary health test workflow and oral microbiome reference database construction.

FIG. 2A-2E illustrates a distribution of the average log ratio difference scores between pairwise microbial interactions associated with healthy cohorts and (A) CKD, (B) struvite crystals or stones, (C) calcium oxalate crystals or stones, (D) cystine crystals or stones, (E) idiopathic cystitis.

FIGS. 3A-3E illustrate sensitivity and specificity of the feline renal/urinary health test based on a 2-component Gaussian mixture model. Sensitivity refers to the ability of the disclosed embodiments to detect cats known to suffer from a renal/urinary condition. Specificity refers to the ability of the disclosed embodiments to detect cats in the healthy control cohorts as not suffering from a renal/urinary condition.

FIG. 4A-B illustrates overlap of oral microbiome predictive microbes characteristic of (A) feline CKD and periodontal disease and (B) feline CKD, struvite urinary crystals or stones, urinary calcium oxalate crystals or stones, cystine urinary crystals or stones, or idiopathic cystitis.

FIG. 5 illustrates microbial species richness as a function of number of sequencing reads, comparing data from two different types of metagenomic whole genome sequencing (WGS) library preparations—a ligation-based approach versus a tagmentation-based approach (such as the Illumina Nextera DNA Flex Library Preparation Kit).

FIG. 6 illustrates an oral microbiome-based CKD risk assessment in citizen science recruited cohorts where clinical records validation of diagnosis was present and the cats were either diagnosed with CKD or considered healthy (no chronic and acute health issues in the last 6 months)

FIG. 7 illustrates an oral microbiome-based CKD risk assessment in five clinically recruited cats where the stage of CKD was known at the time of oral sample collection.

FIGS. 8A-8B illustrate a distribution of the average log ratio difference scores between pairwise microbial interactions associated with healthy cohorts and (A) diabetes mellitus (DM) and (B) inflammatory bowel disease (IBD).

FIGS. 9A-9B illustrate sensitivity and specificity of (A) the feline diabetes mellitus and (B) IBD test based on a 2-component Gaussian mixture model. Sensitivity refers to the ability of the disclosed embodiments to detect cats known to suffer from IBD or diabetes. Specificity refers to the ability of the disclosed embodiments to detect cats in the healthy control cohorts as not suffering from IBD or diabetes.

### DETAILED DESCRIPTION

Variations in the microbial composition of the mouth (i.e., the oral microbiome) may have associations with certain dental and systemic diseases. This research area is still young and studies on human subjects demonstrating these associations in a comprehensive manner have only been published in the last decade or less. Studies on this topic in companion animals, such as cats and dogs, have been limited. Nutritional and environmental factors, as well as present disease states, may play an important role in the dynamic microbial composition of a cat's mouth (i.e., their oral microbiome). With the mouth being the first line of defense from a constant exposure to foreign microbes, the oral microbiome has evolved to be competitive and territorial. It is comprised of microbes that excel at defending their territory and are typically able to avoid being replaced by foreign invaders, including pathogens. However, dysbiosis inducing events such as poor diet, poor dental hygiene, the onset of systemic diseases, or environmental changes, can lead to pathogenic microbes colonizing disproportionately large parts of the oral cavity (and, thus, altering the oral microbiome), which can be associated with pathology. Understanding the composition of the oral microbiome can provide information not only about the health of the oral tissues, but also about the general health of the animal or human. For example, oral microbiome characteristics have been linked with diseases such as Inflammatory Bowel Disease (IBD), various cancers, chronic kidney disease (CKD), among others. The information provided by the state of the oral microbiome may also be used to manage the health and wellbeing of a pet.

The field of oral microbiome research in companion animals has received little focus and it is still in its infancy. Existing studies base their conclusions on small sample sizes and outdated culture-based techniques for querying the microbiome. It is estimated that only around 2% of all existing bacteria can be cultured in the laboratory, meaning that in studies relying on this method for microbial classification, many important microbial organisms will likely be missed, while false emphasis might be placed on particular species, simply because they could be cultured and measured. This problem is compounded by the fact that lab culturing provides a very bacteria-centric view of the microbiome, often ignoring other microorganisms such as fungi, protozoa, archaea and viruses.

Interrogating the oral microbiome of a cat can be accomplished by using an oral (saliva) sample. Saliva sampling kits have gained popularity in recent years as tests for ancestry and microbial infection have become more prevalent. Available direct-to-consumer microbiome tests typically rely on a technique called ‘16S rRNA gene sequencing,’ which utilizes Next Generation Sequencing (NGS). While this technique provides substantially more information than early bacterial culturing efforts, it can only be used for identifying bacterial species (and some archaea) present in the microbiome. In most cases, these tests do not provide sufficient resolution to reliably, and consistently, identify bacteria beyond the genus level of taxonomic classification. Therefore, in most cases, the test results do not provide the exact species or strain of bacteria comprising the microbiome. Thus, data-driven conclusions using these results are vague and rely on approximation. Moreover, it is well-known that the microbiomes of different sites of the body can be composed of viruses, protozoa, and fungal species, in addition to bacteria and archaea. This means that the 16S rRNA gene sequencing approach zooms in on just one part of the microbiome, ignoring the rest. Embodiments of the present disclosure address these and other problems.

Before describing various embodiments of the present disclosure in detail, it is to be understood that this disclosure is not limited only to the specific parameters, verbiage, and description of the particularly exemplified systems, methods, and/or products that may vary from one embodiment to the next. Thus, while certain embodiments of the present disclosure will be described in detail, with reference to specific features (e.g., configurations, parameters, properties, steps, components, ingredients, members, elements, parts, and/or portions, etc.), the descriptions are illustrative and are not to be construed as limiting the scope of the present disclosure and/or the claimed invention. In addition, the terminology used herein is for the purpose of describing the embodiments and is not necessarily intended to limit the scope of the present disclosure and/or the claimed invention.

Presently disclosed are computer systems, systems and methods for the identification, screening, indication, diagnosis, and/or treatment of renal and/or urinary disease states in cats. Embodiments of the disclosed subject matter describe a method for interrogating the oral microbiome of a domestic cat for the purpose of detecting microbe compositional abundance trends associated with renal/urinary diseases in cats. Detecting, identifying and/or quantifying microbe compositional abundance trends enables a practitioner to screen for and/or indicate whether a cat has a particular renal/urinary disease state. Detecting and identifying renal/urinary disease states enables the practitioner and pet owner to treat and delay the disease progression, and in some cases even potentially prevent the future recurrence of the renal/urinary disease state.

Disclosed methods may compare, for example, a cat's oral microbiome to the oral microbiomes of cats reported by their owners and/or a veterinary professional to have been diagnosed with IBD, DM, CKD, struvite urinary crystals or stones, urinary calcium oxalate crystals or stones, cystine urinary crystals or stones, or idiopathic cystitis. The comparison is carried out using a reference database containing defined microbial profiles, associating one or more microbial species and their respective compositional abundance(s) with one or more renal/urinary conditions.

Disclosed systems and methods can comprise a painless oral swab sample collection. Accordingly, the oral microbiome can be surveyed via buccal, supragingival, and/or subgingival sampling. Such sampling does not require anesthetizing the animal and can be performed by the pet owner at their home or by the veterinarian at the clinic. The disclosed systems and methods can potentially serve as an early indicator of renal/urinary disease-associated processes not yet formally diagnosed or presenting with clinical signs. Routine use may enable identification of early-stage renal/urinary diseases, driving more cats to the veterinary office early on and reducing the number of emergency vet visits in the long run. Earlier identification of renal/urinary, inflammatory, and/or endocrine disease states beneficially saves costs in emergency visits and further saves the lives of cats. Earlier identification of one or more disease states also means more treatment options are available when the one or more disease(s) is/are diagnosed and identified.

### Defined Microbial Profiles Contained in the Reference Database

With the mouth being the first line of defense from constant exposure to foreign microbes, the oral microbiome has evolved to be competitive and territorial. It is comprised of microbes that excel at defending their territory and typically resist being replaced by foreign invaders, including pathogens. These microbes are generally present when a cat is healthy and would represent a healthy microbial profile of a cat's oral microbiome. When the cat is suffering from a renal/urinary, inflammatory (e.g., IBD), or endocrine (e.g., DM) condition, the composition of the oral microbiome may be altered by the presence of foreign or pathogenic microbial species and/or altered abundance ratios between different microbes. Such an alteration in the composition of the oral microbiome might be represented by a pathogenic profile. In some cases, the presence of particular foreign and/or pathogenic microbial species, and their abundance relative to other microbes in the oral cavity, is correlated to the cat suffering from a particular renal/urinary condition.

Identification of the particular (one or more) microbial species (and their respective relative abundance(s)) correlated with particular renal/urinary disease states enables pre-diagnostic screening for the renal/urinary disease state in a cat exhibiting the presence of the identified (one or more) microbial species. In other words, identification and/or indication of the renal/urinary disease state may be correlated to the cat exhibiting a particular pathogenic profile.

The gold standard for the comprehensive study of the microbiome is shotgun metagenomic sequencing, which allows capturing complete or near-complete genomes of organisms across all domains of life, not just bacteria and archaea. The gold standard for metagenomic DNA extraction includes a process called bead-beating. It is recommended for complete microbial cell lysis when studying the abundance and composition of the microbiome. The process helps break apart thicker cell walls, such as those of gram-positive bacteria. It is achieved by rapidly agitating samples with grinding media (balls or beads) in a bead beater.

In one exemplary embodiment of the disclosure, the disclosed systems and methods do not use bead-beating for metagenomic DNA extraction and purposefully abandon such a process. The reason for this is that bead-beating can also introduce significant DNA degradation that interferes with downstream sample processing and can therefore lower the quality of the generated metagenomic sequencing library. Since the disclosed systems and methods, according to one embodiment, do not use bead-beating, it is likely that the oral microbiome data in the resulting analyses suffer from under-representation of gram-positive bacteria. Nonetheless, it enables the recognition of disease-characteristic patterns. In some embodiments, the disclosed systems and methods also enable microbial identification and classification down to the species or, in some instances, the strain level, unlike 16S gene sequencing.

In veterinary practice, dental disease, such as periodontal disease, is a common comorbidity in cats suffering from CKD. The reasons why are not well understood, although some theories suggest that with the progression of untreated periodontal disease, pathogenic microbes enter the blood stream through the gingiva and travel to different organs of the body where their presence is associated with pathology. This theory suggests that CKD pathology can often be traced back to untreated periodontal disease. This theory is supported by the fact that some overlap in microbial species important for each of the two conditions is observed. There is also some overlap between the microbial species involved in different feline urinary/renal conditions. However, also identified were a plethora of microbes whose compositional abundance in the oral microbiome are predictive specifically of CKD, struvite urinary crystals idiopathic cystitis. This suggests that there are microbial profiles associated with specific renal/urinary pathologies, in addition to the existence of a core set of microbes associated with renal/urinary diseases in general. This also suggests that there may be microbial profiles associated with other diseases, such as IBD or DM.

Using shotgun metagenomic oral microbiome sequencing of 38,000 domestic cats and compositional data analysis techniques, a comprehensive survey of the feline oral microbiome was executed, identifying 8,344 microbial species present in the feline oral microbiome. Whether a domestic cat included in the shotgun metagenomic sequencing suffered from a particular renal/urinary condition was determined by asking their owner through a survey if the cat had been formally diagnosed by a veterinarian as suffering from a particular renal/urinary condition, an inflammatory condition or an endocrine condition (e.g., IBD, DM, CKD, struvite urinary crystals or stones, urinary calcium oxalate crystals or stones, cystine urinary crystals or stones, or idiopathic cystitis, etc.).

The reference database is a weighted correlation database and contains at least the identified 8,344 microbial species present in the feline oral microbiome. On average, 606 microbial species per cat were identified, 97% of which were classified as bacteria and archaea, 0.27% as DNA viruses (RNA viruses cannot be detected with shotgun metagenomic sequencing), 0.02% as phages and <2% as fungi. The various microbial species identified as being involved in and contributing to a specific renal/urinary disease are compiled into a “defined microbial profile.” The defined microbial profile is a list or collection of identified one or more microbial species and their respective relative abundances known to contribute to and/or be involved in a specific renal/urinary disease condition. In some embodiments, defined microbial profiles may include percentages of gram-positive microbes and ratios of gram-positive microbes to gram negative microbes, in addition to the identities (i.e., genus and species) of microbes. In some embodiments, defined microbial profiles may indicate the relative abundance (increased or decreased) of the one or more microbial species. (See Tables 1-16 below).

For example, a defined microbial profile may include a set of 38 microbes that are predictive for five renal/urinary conditions (CKD, struvite urinary crystals or stones, urinary calcium oxalate crystals or stones, cystine urinary crystals or stones, idiopathic cystitis), as well as microbes specifically predictive for one of the five renal/urinary conditions (CKD, struvite urinary crystals or stones, urinary calcium oxalate crystals or stones, cystine urinary crystals or stones, idiopathic cystitis). “Predictive microbes” are discussed more fully below. The defined microbial profile may rank and/or weigh each included microbial species by how frequently and in what proportions a certain microbe is observed in felines suffering from the specific renal/urinary condition, as deduced by consulting a reference database. How much any one microbial species contributes to a specific renal/urinary disease condition is correlated to how often a microbial species shows up (or is present) in the oral microbiome while a feline is suffering from a specific renal/urinary disease condition. How much any one microbial species contributes to a specific renal/urinary disease condition is also correlated to how consistently such microbial species demonstrates significantly different relative abundance from other oral microbes when compared to healthy control samples.

The defined microbial profiles contained in the reference database also include defined microbial profiles of healthy cats that are not suffering from a renal/urinary condition. For example, the defined microbial profile of healthy cats lists and identifies the microbial species present in the oral microbiome, as well as their relative abundances, when no renal/urinary condition is present. A healthy defined microbial profile may establish a baseline or control for the microbial species present and their relative abundances. Any deviations from this profile may enable a practitioner to predict and/or indicate, for example, a cat's likelihood of suffering from a renal/urinary condition. Similarly, deviations from the healthy defined microbial profile may enable a practitioner in diagnosing a cat as suffering from a renal/urinary condition prior to the onset of symptoms for that renal/urinary condition.

The defined microbial profile for each renal/urinary disease state is compared to the defined microbial profile for a healthy cat to determine any differences between the renal/urinary disease states and a healthy state. In some embodiments, the comparisons are pairwise log ratio comparisons. For example, there may be some overlap in the oral microbiome of a healthy cat and a cat suffering from CKD. A comparison of the healthy defined microbial profile to the CKD defined microbial profile would identify common microbial species seen in similar abundances between the two. Any microbial species not common between the two microbial profiles, or any microbial species seen in significantly different proportions between the two profiles, would confirm the involvement of that microbial species in the development of CKD. Identification of such a microbial species in a cat's oral microbiome would be indicative of the cat having CKD.

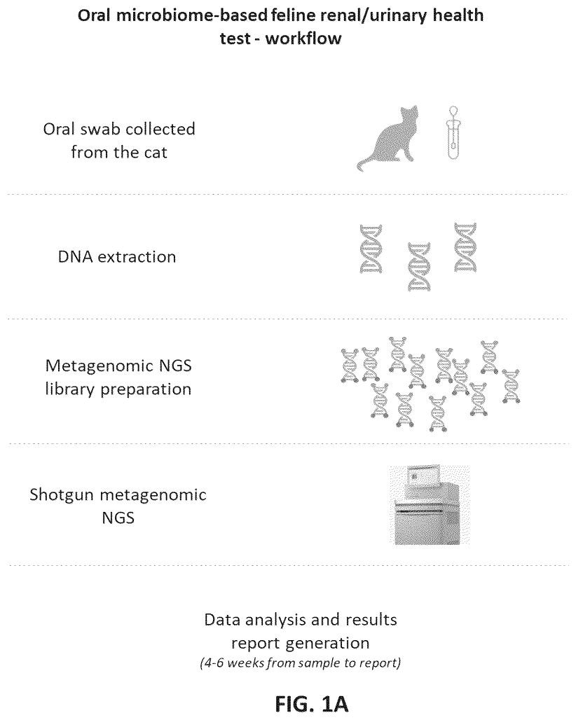

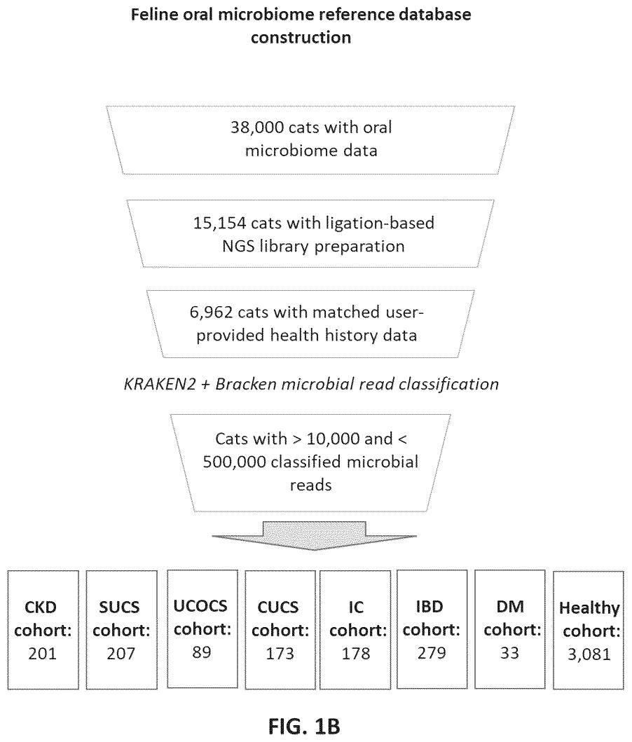

FIGS. 1A-1B illustrate a renal/urinary health test workflow and construction of the oral microbiome reference database using feline subjects. In FIG. 1A, the feline renal/urinary health test workflow includes collecting an oral swab from the cat in a DNA preservation solution, extracting and preparing the DNA for shotgun metagenomic next generation sequencing (NGS), sequencing the DNA, data analysis, and the generation of a report presenting risk assessment for different renal/urinary diseases based on the state of the oral microbiome, accompanied by treatment recommendations tailored to the results. In FIG. 1B, the feline oral microbiome reference database was constructed through applying sequential filters on the initial database of 38,000 cats. First, all data from tagmentation-based NGS library preparation samples was removed. This was done due to an observed effect of the library preparation method on microbial species richness (see FIG. 5). The ligation-based method was preferred because the number of sequencing reads per sample had minimal impact on the number of microbial species detected. In addition, Tn5 transposase assisted tagmentation is known to introduce GC sequencing bias, particularly in metagenomic communities. However, tagmentation-based NGS library preparation may be included in some embodiments.

Next, samples lacking an accompanying relevant phenotype/health history record for the cat were excluded. The microbial sequence data from the metagenomic sequence data of the sample is identified, classified, and mapped. After classification of the microbial reads in each sample using KRAKEN2 and Bracken, samples with fewer than 10,000 and more than 500,000 classified microbial reads were removed. The remaining cats/samples were placed into cohorts. This resulted in a chronic kidney disease cohort (CKD; n=201), struvite urinary crystals or stones cohort (SUCS; n=207), urinary calcium oxalate crystals or stones cohort (UCOCS; n=89), cystine urinary crystals or stones cohort (CUCS; n=109), idiopathic cystitis cohort (IC; n=178) and a healthy cohort (n=3,081).

Though FIGS. 1A-1B illustrate a renal/urinary health test workflow and construction or the oral microbiome reference database, it is to be understood that the same health test workflow was performed for inflammatory conditions (e.g., IBD) and endocrine conditions (e.g., DM). Thus, an IBD cohort (n=279) and a DM cohort (n=33) were obtained, classified, mapped, and, likewise, added to the oral microbiome reference database. Use of the oral microbiome reference database in conjunction with the disclosed computer systems, systems and methods enables a practitioner to screen for, indicate, identify, diagnose, and/or treat disease states in cats. The disease states include, at least, IBD, DM, CKD, SUCS, UCOCS, CUCS, and IC.

### Identifying Predictive Microbes

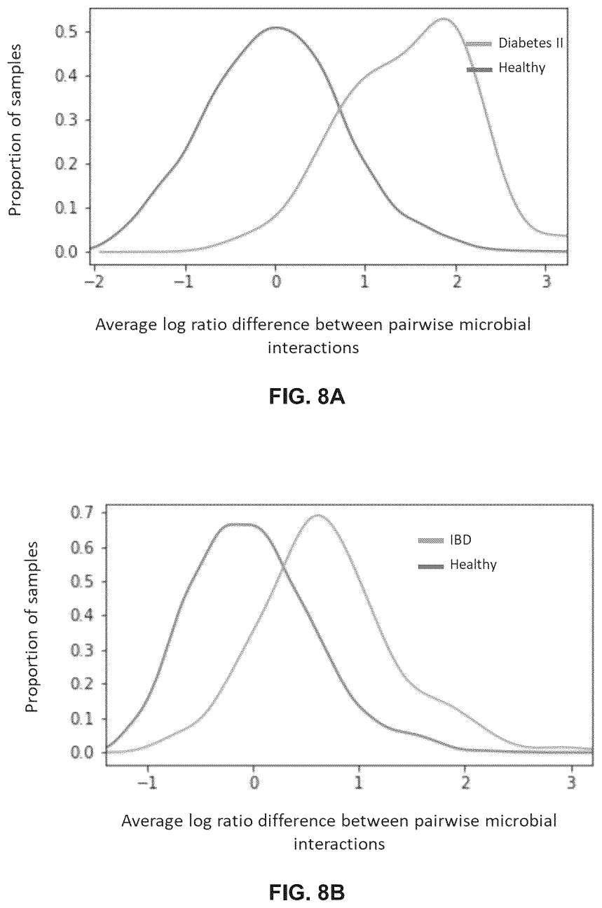

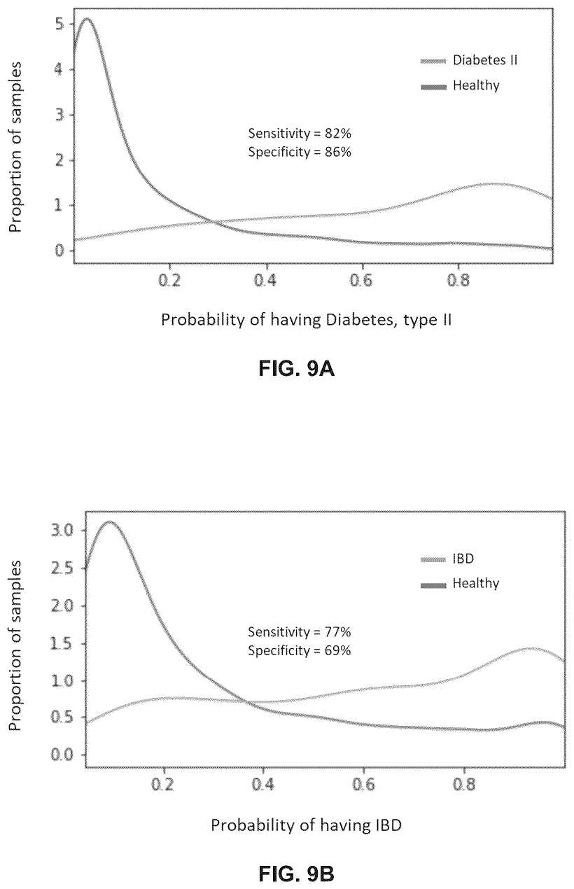

As a first step towards identifying microbes significantly correlated with each renal and/or urinary condition, Pairwise Log-Ratio (PLR) transformation was performed on the Bracken output species level read counts. Next, the significant PLR comparisons (p-value<0.01) were identified between the control (i.e., healthy cohort) and a condition by performing a z-test. The healthy cohort was compared to the CKD, SUCS, UCOCS, CUCS and IC cohorts. The healthy cohort was also compared to an IBD cohort and DM cohort. (See FIGS. 8A-9B).

The frequency of each microbial species in all significant PLRs was assessed. Only microbial species where 50% or more of their maximum possible comparisons with other species were significant were kept. This measure was used as a proxy for the importance of different microbial species in the five renal/urinary conditions of interest. These microbial species are “predictive microbial species” for each renal/urinary condition.

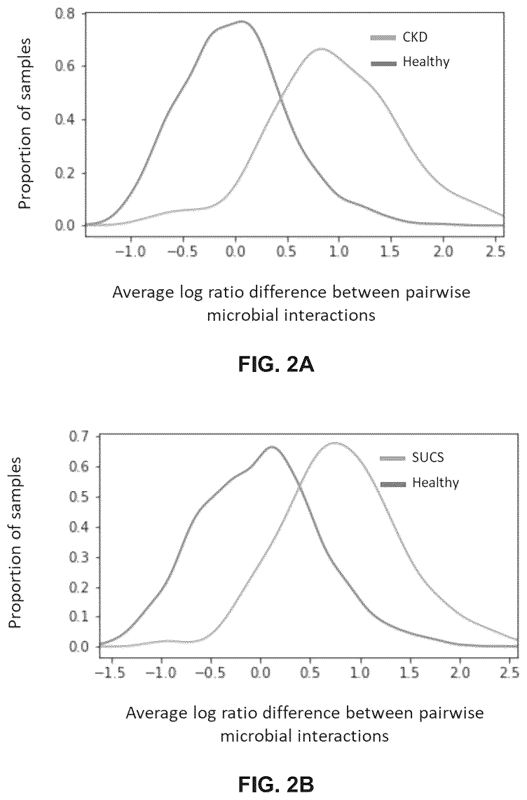

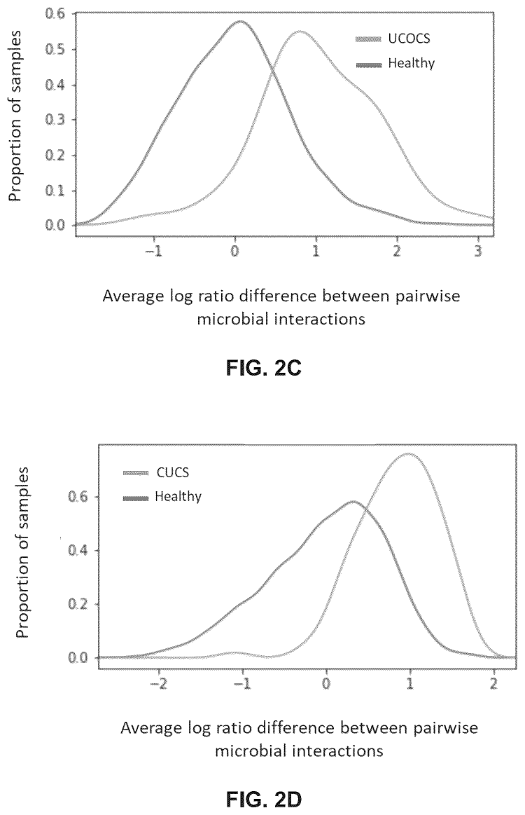

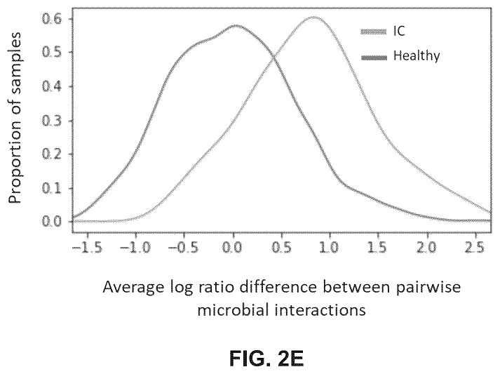

In order to identify population-wide microbial compositional abundance patterns characteristic of CKD, struvite urinary crystals or stones, urinary calcium oxalate crystals or stones, cystine urinary crystals or stones, and idiopathic cystitis, each sample was scored by comparing the predictive pairwise log-ratios (pPLRs) of the sample to the mean pPLRs of controls, taking into account the direction and magnitude of the difference. FIGS. 2A-2E illustrate a distribution of the average log ratio difference scores between pairwise microbial interactions associated with CKD, struvite urinary crystals or stones, urinary calcium oxalate crystals or stones, cystine urinary crystals or stones and idiopathic cystitis and healthy cohorts.

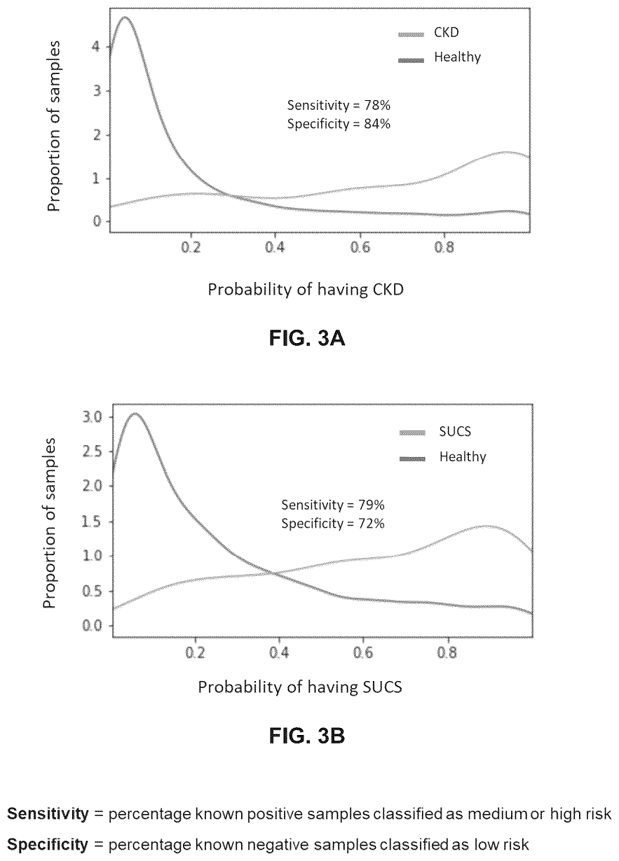

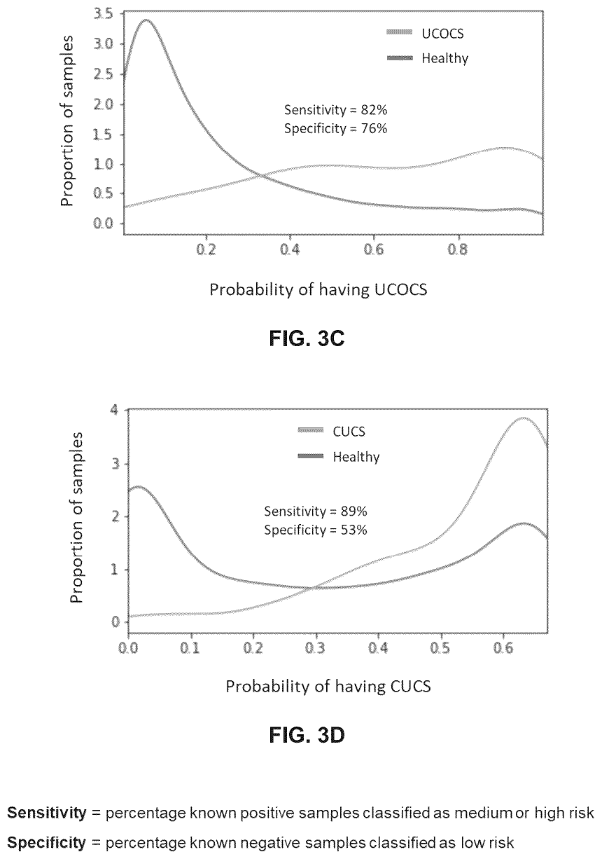

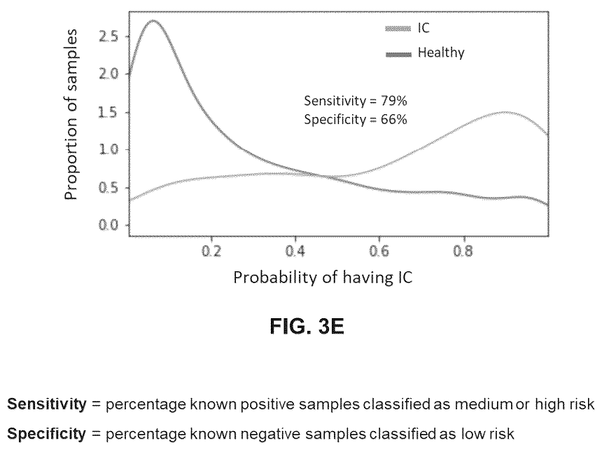

Next, we fitted five (5) Gaussian mixture models (one for each renal/urinary condition) with two (2) components each—healthy cohort and urinary and/or renal condition—onto the distribution of the average log ratio difference score between pairwise microbial interactions. This modeling approach generates a 0 to 1 score for each sample, which represents the probability that the sample belongs to the control cohort or to the respective renal/urinary condition cohort. FIGS. 3A-3E plot the probability that samples belonging to five of the renal/urinary disease cohorts (CKD, struvite urinary crystals or stones, urinary calcium oxalate crystals or stones, cystine urinary crystals or stones, and idiopathic cystitis) and the control samples would be classified as belonging to their respective cohorts based on each sample's compositional abundance of predictive microbes. A bimodal probability distribution consistent with sample identity was observed between the renal/urinary condition and control in all cases. There was a minority of disease samples forming a small peak closer to 0 and a small set of control samples forming a slight peak closer to 1.

The defined microbial profile for each renal/urinary disease state (CKD, struvite urinary crystals or stones, urinary calcium oxalate crystals or stones, cystine urinary crystals or stones and idiopathic cystitis) is compared to the defined microbial profile for a healthy cat to determine and quantify differences and commonalities in microbial species and their abundance between the renal/urinary disease states and a healthy state. The defined microbial profiles for each renal/urinary disease state are also compared to each other to identify overlapping microbial species common to each renal/urinary disease state. The defined microbial profiles for IBD and DM underwent similar comparisons to determine and quantify differences and commonalities in microbial species and their abundance between IBD/DM and a healthy state, as well as to identify overlapping microbial species common to each disease state.

The defined microbial profiles for each disease state and a healthy control state undergo a pairwise log ratio (PLR) transformation. The PLR transformation corrects for potential sequencing coverage differences between samples by scaling microbial abundances relative to each microbe instead of a constant scaling factor. Next, a z-test between PLRs from each disease state versus the control state is performed. A p-value of approximately <0.01 serves as a threshold value for significant PLR comparisons. For each microbial species identified in a defined microbial profile for a renal/urinary disease state, the number of significant PLR comparisons (as defined by the p-value) that microbial species shows up in is counted. If the number of significant PLR comparisons is at least 50% of all possible PLR comparisons for that microbe, the microbial species is deemed a “predictive microbe.” This process may be repeated for each renal/urinary disease state of interest. In other words, through z-test identification of significant PLR comparisons, predictive microbes can be identified for IBD, DM, CKD, struvite urinary crystals/stones, urinary calcium oxalate crystals/stones, cystine urinary crystals/stones and idiopathic cystitis. Table 1 provides examples of identified predictive microbes for CKD, struvite urinary crystals or stones, urinary calcium oxalate crystals or stones, cystine urinary crystals or stones, and idiopathic cystitis. Table 2 provides examples of identified predictive microbes for IBD and DM.

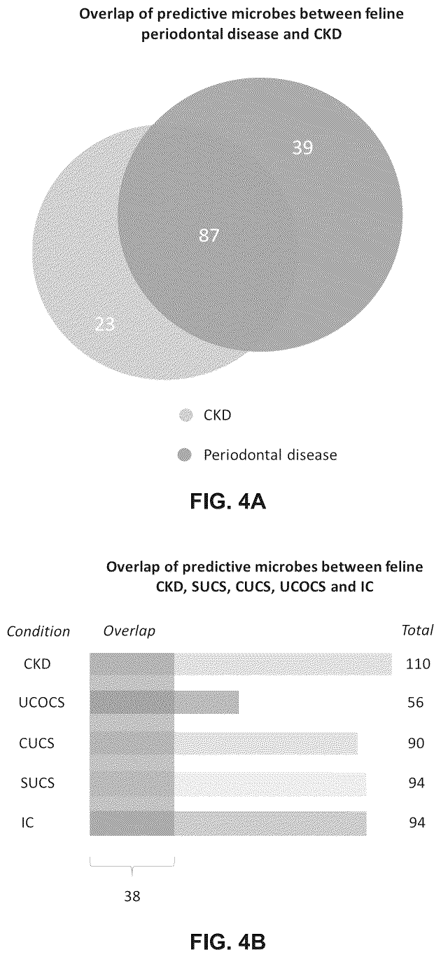

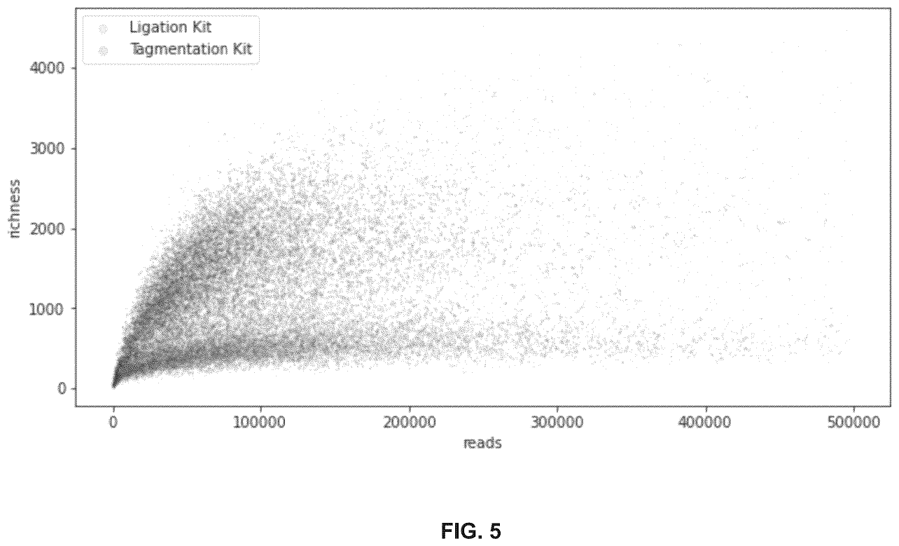

As outlined in Table 1, 110 predictive microbes for CKD, 94 for struvite urinary crystals or stones, 56 for urinary calcium oxalate crystals or stones, 90 for cystine urinary crystals or stones, and 94 for idiopathic cystitis were identified. The predictive microbes for each renal/urinary disease were identified based on PLR microbial abundance comparisons between healthy/control defined microbial profiles and the defined microbial profiles of cats suffering from one of five renal/urinary conditions (See FIG. 4). 38 microbes were identified as predictive for the five renal/urinary conditions (CKD, struvite urinary crystals or stones, urinary calcium oxalate crystals or stones, cystine urinary crystals or stones, and idiopathic cystitis), though each condition has its own specific set of predictive microbes, differentiating it from other conditions. Plotting the average log ratio difference between significant pairwise microbial interactions in a renal/urinary condition versus control samples allowed separation of sample populations based on their renal/urinary disease status. (See FIGS. 2A-2E). However, some overlap between the populations was observed, meaning that for a certain set of samples, their compositional abundance of predictive microbes could be interpreted as either consistent with the control population or the respective renal/urinary disease population.

Tables 3-9 outline the percentages of microbes identified or associated with the various disease states of interest (e.g., IBD, DM, CKD, struvite urinary crystals or stones, urinary calcium oxalate crystals or stones, cystine urinary crystals or stones, and idiopathic cystitis). Tables 10-16 outline the relative increased or decreased abundance for each predictive microbe for each disease state of interest. This data (regarding relative abundances, percentages, and ratios of gram-positive bacteria present) may also be included in the defined microbial profiles for each disease state. Detection of one or more gram-positive bacteria (or, obtaining a ratio or percentage of one or more of these gram-positive bacteria) in the oral microbiome of a cat may enable the systems and methods to indicate or diagnosis the cat as suffering from a specific disease (e.g., IBD, DM, CKD, struvite urinary crystals or stones, urinary calcium oxalate crystals or stones, cystine urinary crystals or stones, and idiopathic cystitis).

The same process (comparison, PLR transformations, z-test, etc.) was performed for IBD and DM cohorts. FIGS. 8A-8B illustrates a distribution of the average log ratio difference scores between pairwise microbial interactions associated with healthy cohorts and (A) diabetes mellitus (DM), and (B) inflammatory bowel disease (IBD). FIGS. 9A-9B illustrate sensitivity and specificity of the feline IBD and diabetes mellitus health test based on a 2-component Gaussian mixture model. Table 2 lists the predictive microbes associated with IBD and DM. Tables 3 and 4 outline the percentage of gram-positive predictive bacteria identified or associated with DM and IBD, respectively, alongside the disease-specific breakdown of predictive microbes falling under different taxonomic classifications (different genera of bacteria, as well as fungi and viruses). Tables 10 and 11 outline the relative increased or decreased abundance for each predictive microbe for DM and IBD, respectively.

It is important to note that the use of the word ‘predictive’ is not meant to be interpreted as ‘causative’, it simply reflects the fact that a microbe has a significantly different compositional abundance in a particular renal/urinary condition compared to control. This could either mean that the microbe has an active role in the disease's pathology or that the changes of its compositional abundance are a byproduct of pathology. In either scenario, presence of the microbe in a specific abundance relative to other microbes directly correlates with a renal/urinary disease state.

The algorithms and disclosed methods of identifying predictive microbes may be continually evolving. A set or grouping of identified predictive microbes may slightly change and evolve as the populations of the cohorts (healthy cats and cats with a renal or urinary condition) change and evolve. As more information becomes available regarding microbes and their presence or contribution to a renal/urinary, inflammatory, or endocrine disease state, the set of identified predictive microbes will change and evolve. The new set of identified predictive microbes may not be 100% different from the initial set, rather a variance of approximately 25% to 85% may be expected. For example, the new set of identified predictive microbes may be 30, 35, 40, 45, 50, 55, 60, 65, 70, 75, or 80% different from the initial set of identified predictive microbes, or a variance defined by any two of the foregoing values. As more cats are added to the cohorts, the set of identified predictive microbes will change and evolve.

### Sequencing and Extraction Protocols

At least one oral swab of a cat may be taken to provide a sample for testing. The oral swabs may target the gum lines of the animal (top and bottom) and/or target the entire mouth of the animal. Microbial DNA may be extracted from the oral swab samples in order to identify which microbial species, and in what relative abundance, are present in the cat's oral microbiome.

Metagenomic DNA may be extracted from the oral samples via heat treatment for approximately one hour on a shaker, with or without bead-beating or the addition of detergents and protein degradation reagents such as proteinase K. In some embodiments, the oral samples are heat treated at approximately 45° C. to 75° C., such as 50° C., 55° C., 60° C., 65° C., 70° C. or within a range defined by any two of the foregoing values.

The gold standard for the comprehensive study of the microbiome is shotgun metagenomic sequencing, which allows capturing complete or near-complete genomes of organisms across all domains of life, not just bacteria and archaea. The gold standard for metagenomic DNA extraction includes a process called bead-beating. It is recommended for complete microbial cell lysis when studying the abundance and composition of the microbiome. The process helps break apart thicker cell walls, such as those of gram-positive bacteria. It is achieved by rapidly agitating samples with grinding media (balls or beads) in a bead beater.

In one exemplary embodiment, the disclosed systems and methods do not use bead-beating for metagenomic DNA extraction and purposefully abandon such a process. The reason for this is that bead-beating can also introduce significant DNA degradation that interferes with downstream sample processing and can therefore lower the quality of the generated metagenomic sequencing library. Since the disclosed systems and methods, according to one embodiment, do not use bead-beating, it is likely that the oral microbiome data in the resulting analyses suffers from under-representation of gram-positive bacteria. Nonetheless, it enables the recognition of disease-characteristic patterns.

After heat treatment of the oral sample, metagenomic DNA may be extracted by SPRI magnetic beads-based DNA extraction (MCLAB, MBC-200) using 80% ethanol for purification. The DNA may be quantified using a GloMax Plate Reader (Promega). Following metagenomic DNA extraction and quantification, the oral samples may be prepared for NGS using the LOTUS DNA library prep kit (IDT), the Next Ultra II FS DNA library prep kit (NEB), or another ligation or tagmentation based DNA library prep kit, following the manufacturer's instructions. The oral samples may be dual-barcoded with iTRU indices. The prepared sequencing libraries may be quantified using a GloMax Plate Reader (Promega) and equal-mass pooled into 96-sample pools. The pools may then be visualized (to assess fragment size distribution) and quantified using a 2100 Bioanalyzer instrument (Agilent). Following standard QC steps, the 96-sample pools may be loaded onto an Illumina HiSeq X or NovaSeq 6000 Next Generation Sequencing machine.

The raw sequencing data may be demultiplexed and trimmed to remove low-quality data using, for example, the program Trimmomatic 0.32. The data may then be mapped to the latest version of, for example, the feline genome Felis_catus_9.0. For every oral sample, there may be approximately 5-7% sequencing reads that do not map to the feline genome. The unmapped reads may be classified using the KRAKEN2 metagenomic sequence classifier (or a suitable alternative) to identify the microbial organisms present in each sample. Bracken, a statistical method for calculating species abundance in DNA sequencing data from a metagenomic sample, may be used on the sequenced data in conjunction with the KRAKEN2 analysis. Bracken may output species level read counts. Based on the outcome of the KRAKEN2 metagenomic sequence classifier and the Bracken calculations, an oral microbial profile for the cat may be generated. The oral microbial profile generated may include data regarding the identity of the microbial species present as well as their relative abundance. The oral microbial profile generated may also include data regarding the percentage of gram-positive bacteria.

A confidence score of approximately 0.1 (e.g., 0.08 to 0.15) may be used as a cutoff (or threshold value) for the KRAKEN2 classification algorithm. All samples with fewer than 10,000 classified microbial reads or more than 500,000 classified microbial reads may be filtered out. The reads for microbial species with a non-zero mean of fewer than 10 reads may also be filtered out.

### Methods of Indication and Comparison

Indication of whether a cat is suffering from one or more diseases (e.g., a renal/urinary disease, an inflammatory disease, or an endocrine disease) relies on a comparison of the cat's current oral microbiome state to the oral microbiomes of cats reported by their pet owners to have been diagnosed by a veterinarian with IBD, DM, CKD, struvite urinary crystals idiopathic cystitis. The comparison is based on the compositional abundance of microbes determined by the analysis to be predictive of each of the conditions.

Computational analysis of the compositional abundance of different microbes present in the oral microbiome involves comparison of the sample against a database of samples from cats known to suffer from the different conditions, as well as cats who do not suffer from any known renal/urinary, IBD, or DM conditions. In other words, the computational analysis compares the oral microbiome identified from the oral swab sample to the defined microbial profiles contained in the reference database (discussed more fully above).

In some embodiments, a method for indicating renal/urinary disease in cats includes receiving an oral swab sample taken from a cat; performing heat treatment on the oral sample; and performing magnetic beads-based deoxyribonucleic acid (DNA) extraction on the heat-treated oral sample to extract microbial DNA that is present in the oral swab sample. The method may also include sequencing the microbial DNA to identify which specific one or more microbes are present in the oral sample and in what proportions (i.e., abundance), wherein identifying the specific one or more microbes and their abundances results in generation of an oral microbial profile for the cat; and comparing the oral microbial profile for the cat against a database of defined microbial profiles, wherein the database identifies correlations between (i) profiles that include one or more microbes and (ii) corresponding renal/urinary diseases.

Based on a result of comparing the oral microbial profile for the cat against the database of defined microbial profiles, the method may further include generating a risk score indicating a likelihood that the cat has a specific renal/urinary disease. The risk score may be correlated to a stage or severity of the disease state (e.g., a higher risk score associated with stage 2 CKD).

In some embodiments, a method for indicating renal/urinary disease in cats includes receiving an oral swab sample taken from a cat; performing heat treatment on the oral sample; and performing magnetic beads-based deoxyribonucleic acid (DNA) extraction on the heat-treated oral sample to extract microbial DNA that is present in the oral swab sample. The method may also include sequencing the microbial DNA to identify which specific one or more microbes are present in the oral sample, wherein identifying the specific one or more microbes and their abundance results in generation of an oral microbial profile for the cat.

The method may further include comparing the oral microbial profile for the cat against a database of defined microbial profiles, wherein the database identifies correlations between (i) profiles that include one or more microbes and (ii) corresponding renal/urinary diseases; based on a result of comparing the oral microbial profile against the database of defined microbial profiles, generating a risk score indicating a likelihood that the cat has a specific renal/urinary disease; and in response to generating the risk score and identifying the specific renal/urinary disease, administering a therapeutic treatment designed to treat the specific renal/urinary disease.

In some embodiments, the therapeutic treatment may include administering a therapeutic compound, such as a compound designed to inhibit or encourage growth of a specific one or more microbial species present in the oral microbiome of the cat. In some embodiments, the therapeutic compound includes a pre-biotic, a post-biotic, a pro-biotic, a medicament or a combination thereof. In some embodiments, the therapeutic treatment may include brushing the cat's teeth with a topical treatment.

In some embodiments, the therapeutic compound includes a phosphate binder, an antibiotic, a compound to control hypertension and/or blood pressure of the cat, and erythropoietin, among other therapeutic compounds. In some embodiments, the therapeutic treatment may include a dietary regimen designed to address and/or alleviate the renal/urinary disease state. For example, therapeutic diets that are restricted in protein, phosphorus and sodium content, and high in water-soluble vitamins, fiber, and antioxidant concentrations, may prolong life and improve quality of life in cats with CKD. In some embodiments, the dietary regimen may include switching to a wet food to help maintain proper hydration of the cat. The therapeutic treatment may include potassium supplementation.

In some embodiments, the therapeutic treatment protocol is designed to alter the composition of the oral microbiome of the cat. In some embodiments, altering the composition of the cat's oral microbiome treats and/or addresses the specific renal/urinary disease. In some embodiments, the therapeutic treatment repairs the cat's oral microbiome. In some embodiments, repairing the cat's oral microbiome brings the cat's oral microbiome more in line with the oral microbiome (or defined oral microbial profile) of a healthy cat-both in terms of the specific one or more microbial species present and their relative abundance. In some embodiments, the therapeutic treatment protocol is designed to maintain the composition of the oral microbiome of the cat. In some embodiments, the therapeutic treatment protocol is designed to stimulate a metabolic output of the cat's oral microbiome. Stimulating a metabolic output of the cat's oral microbiome may include using known enzymatic pathway analysis tools to provide an additional dimension to the existing microbial composition data to further characterize disease signatures and improve predictive disease models.

### Examples

To start building computational renal/urinary, inflammatory, and endocrine disease classification algorithms, Pairwise Log-Ratio (PLR) transformation was performed on the Bracken output species level read counts. Bracken is a statistical method for calculating species abundance in DNA sequencing data from a metagenomic sample. Next, the significant PLR comparisons (with a threshold p-value<0.01) were identified between the control and a condition by performing a z-test. The transformed data may be stored in the database. The healthy cohort was compared to the CKD, SUCS, UCOCS, CUCS and IC cohorts. The healthy cohort was also compared to the IBD and DM cohorts. (See FIGS. 8A-9B).

The frequency of each microbial species in all significant PLRs was assessed. Only microbial species where 50% or more of their maximum possible comparisons with other species were significant were kept. This measure was used as a proxy for the importance of different microbial species in the five renal/urinary disease conditions, the inflammatory condition (IBD) and the endocrine condition (DM) of interest. These microbial species are “predictive microbial species” for each renal/urinary condition.

In order to identify population-wide microbial compositional abundance patterns characteristic of CKD, struvite urinary crystals or stones, urinary calcium oxalate crystals or stones, cystine urinary crystals or stones, and idiopathic cystitis, for each of the conditions, each sample was scored by comparing the predictive pairwise log-ratios (pPLRs) of the sample to the mean pPLRs of controls, taking into account the direction and magnitude of the difference.

FIGS. 2A-2E illustrate a distribution of the average log ratio difference scores between pairwise microbial interactions associated with CKD and healthy cohorts, struvite urinary crystals or stones and healthy cohorts, urinary calcium oxalate crystals or stones and healthy cohorts, cystine urinary crystals or stones and healthy cohorts, and idiopathic cystitis and healthy cohorts. FIGS. 8A-8B illustrate a distribution of the average log ratio difference scores between pairwise microbial interactions associated with DM and healthy cohorts, and IBD and healthy cohorts.

Next, we fitted five (5) Gaussian mixture models (one for each renal/urinary condition) with two (2) components each—healthy cohort and renal/urinary condition-onto the distribution of the average log ratio difference score between pairwise microbial interactions. This modeling approach generates a 0 to 1 score for each sample, which represents the probability that the sample belongs to the control cohort or to the respective renal/urinary condition cohort. FIGS. 3A-3E plot the probability that samples belonging to five of the renal/urinary disease cohorts and the control samples would be classified as belonging to their respective cohorts based on each sample's compositional abundance of predictive microbes. A bimodal probability distribution consistent with sample identity was observed between renal/urinary condition and control in all cases. In all five instances, there was a minority of disease samples forming a small peak closer to 0 and a small set of control samples forming a slight peak closer to 1. FIGS. 9A-9B plot the probability that samples belonging to the IBD or DM cohorts and the control samples would be classified as belonging to their respective cohorts based on each sample's compositional abundance of predictive microbes.

This suggests that it is possible that a small proportion of cats in the renal/urinary disease cohorts, the IBD cohorts and the DM cohorts might actually be healthy or in remission (due to old, wrong or incomplete health information provided by the pet owner), while some cats in the control cohorts could be suffering from a renal/urinary, inflammatory or endocrine condition that has not yet been diagnosed or noticed. The sensitivity (ability to detect cats known to suffer from a condition) and specificity (ability to detect cats in the control cohort as not suffering from a condition) of the risk classification method for each condition was tested (see FIGS. 3A-3E and 9A-9B). The method's sensitivity is highest for cystine urinary crystals or stones and lowest for IBD, while the specificity is highest for DM and lowest for cystine urinary crystals or stones.

Even though a sizable domestic cat cohort (n=3,929) was used to develop the reference database, the health history data for these cats was provided by the pet owner. Despite the fact that pet owners were asked if their cats had been diagnosed by a veterinarian with CKD, SUCS, CUCS, UCOCS, or IC, some of the diagnostic precision would have, undoubtedly, suffered, having been relayed by the pet owner. To alleviate this problem and limit instances where a cat reported by their pet owner to be healthy (i.e., not suffering from any known systemic or renal/urinary conditions) had actually started developing a yet undiagnosed disease, an age limit was set to the control healthy cohort of 1-3 years. This limit was set due to the well-established connection between age and renal/urinary and systemic disease. Cats below one year of age were intentionally excluded from this group with the purpose of avoiding any potential kitten-specific oral microbiome bias. The healthy control cohort could potentially be biased towards the oral microbiomes of younger cats and not be representative of older cats with no renal/urinary or systemic diseases

Though an age limit was set to the control healthy cohort, some embodiments of the present disclosure do not set an age limit. In some embodiments, age of the cat is included as a factor in identifying the cat's risk for having or developing a renal/urinary disease condition. In some embodiments, age may impact the grouping of the cohorts, with older cats being in a separate cohort from younger cats, even for the same renal/urinary condition. In some embodiments, age is a factor applied to a cat's risk assessment after comparison of the cat's oral microbial profile to the cohorts (healthy and pathological). In some embodiments, age is incorporated into the oral microbial profile obtained and generated for the cat.

In addition to the foregoing, microbes identified as associated with or predictive for a condition may be further predictive for stages or grades of the condition. For example, a subset of the predictive microbes for CKD can be indicative of stage 2 CKD. Early detection of the stage of a disease enables broader treatment options. Thus, use of a subset of predictive microbes for earlier detection of the stage of the disease benefits cats and owners by driving unhealthy cats to the clinic before the disease progresses beyond treatment. It also benefits veterinarians by enabling them to better select a treatment option based on the stage or grade of the condition.

### Study 1

Following obtainment of written consent from pet owners (over email), 32 feline oral swab samples from cats suffering from various stages of CKD were collected, where samples were taken by the pet owner at their home using DNAGenotek PERFORMAGENE P-100 collection devices. The same approach was used for the collection of oral swab samples from 15 healthy cats. Each cat participating in this trial had accompanying up-to-date veterinary records. To be accepted in the trial, cats in the CKD cohort had to have a clinical record clearly stating a CKD diagnosis, while cats in the healthy cohort had to have a clinical record within the last six months demonstrating the absence of any diagnosed chronic or acute disorders.

DNA was extracted from these samples, after which shotgun metagenomic sequencing was performed and the data was analyzed using the computational renal/urinary disease risk assessment methods and/or computer systems described previously. The algorithm produced CKD risk assessments for these two cohorts.

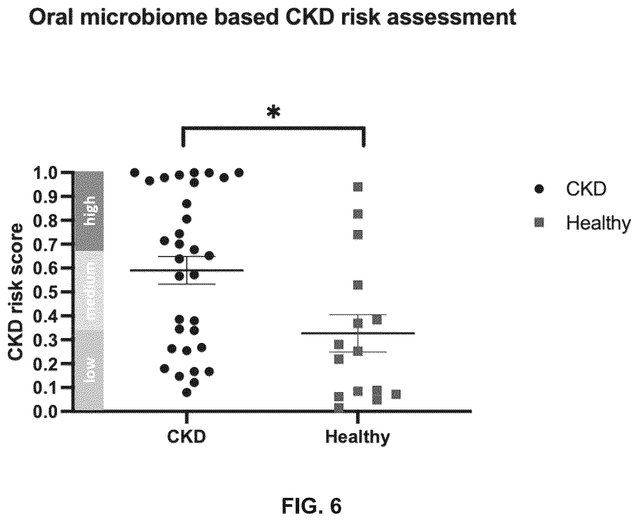

The average generated oral microbiome based CKD risk assessment (i.e., risk score) was significantly higher for the CKD cohort compared to the healthy cohort (p<0.05). FIG. 6 illustrates these findings. The horizontal lines represent the mean risk score for each cohort (the risk score range is from 0 to 1, with higher values representing increased risk of disease) and the error bars represent the Standard Error of the Mean (SEM). A 2-tailed t-test assuming unequal variance was used for each comparison; *p<0.05.

### Study 2

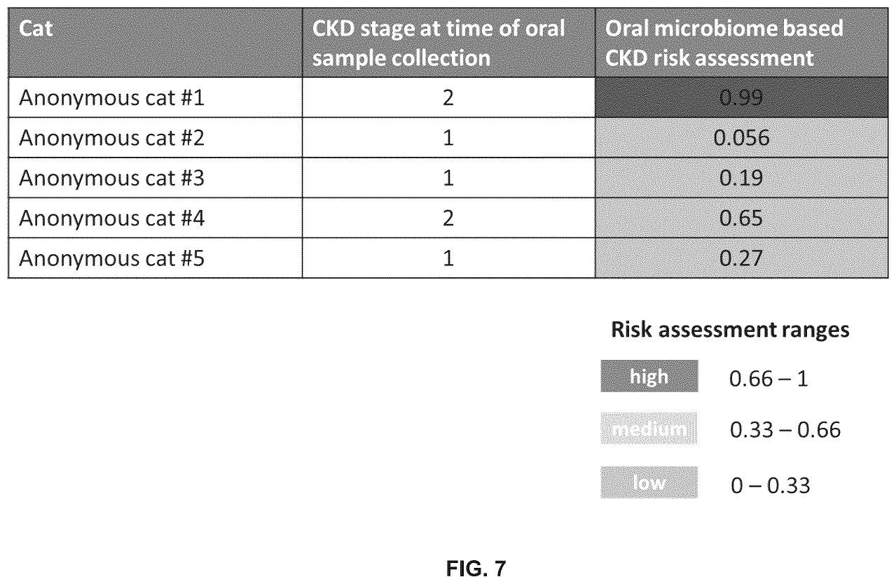

Following obtainment of written consent from pet owners, oral swab samples were collected during a veterinary visit by a licensed veterinary technician from cats diagnosed with stage 1 or stage 2 CKD. DNAGenotek PERFORMAGENE P-100 collection devices were used. DNA was extracted from these samples, after which shotgun metagenomic sequencing was performed and the data was analyzed using the computational renal/urinary disease risk assessment methods and/or computer systems described previously. The algorithm produced CKD risk assessments for each sample. The algorithms classified stage 1 CKD cats as low risk for the disease and stage 2 CKD cats as medium or high risk for the disease. The results are summarized in FIG. 7.

In addition to the foregoing, microbes identified as associated with or predictive for CKD, for example, are further predictive for stages or grades of CKD. For example, a subset of the predictive microbes for CKD can be indicative of stage 2 CKD. Early detection of the stage of a renal/urinary disease enables broader treatment options. Thus, use of a subset of predictive microbes for earlier detection of the stage of the renal/urinary disease benefits cats and owners by driving unhealthy cats to the clinic before the disease progresses beyond treatment. It also benefits veterinarians by enabling them to better select a treatment option based on the stage or grade of the condition. Similarly, use of a subset of the predictive microbes for IBD and DM may also be indicative of varying stages or severity of the conditions.

### Discussion

Many inflammatory, endocrine, renal and urinary diseases progress through stages or grades. Conditions like IBD are known to get progressively worse and harder to treat with the onset of more severe symptoms. CKD is typically associated with four stages, with stage 3 typically being the stage at which cats are formally diagnosed with the disease. To formally diagnose stages 1 and 2 of CKD, veterinarians may conduct a physical examination and run blood work or other tests. In the physical examination, a veterinarian may look for palpable kidney abnormalities, evidence of weight loss, dehydration, pale mucous membranes, uremic ulcers, and evidence of hypertension (i.e., retinal hemorrhages/detachment). Veterinarians may also measure symmetric dimethylarginine (SDMA) levels in the blood as SDMA is regarded as an early detection blood marker.

To formally diagnose the later stages of CKD (i.e., 3 and 4), the veterinarian may measure creatine and SDMA levels in the blood. The specific gravity of a cat's urine may also be measured as part of diagnosis. Based on the stage of the disease, the treatment protocol may differ. For example, when diagnosed at stage 1 CKD, there are many treatment and preventative options. Among other things, trends in SDMA and creatine levels may be monitored, the diet may be modified to manage hypertension and phosphorous levels, and investigation of underlying causes may be undertaken. As the cat progresses through the various stages of CKD, the treatment options may change.

The disclosed methods and systems were successfully used to distinguish cats diagnosed with CKD from cats that had not been diagnosed with any renal/urinary or systemic diseases. While Study 1 used citizen science recruited feline oral samples, every sample's disease status was confirmed by the cat's clinical records. The disclosed algorithm produced a significantly higher average CKD risk assessment (i.e., risk score) for the cats that had been diagnosed with CKD compared to the CKD risk assessment produced for healthy cats. The fact that in Study 1 a minority of CKD samples were classified as low risk and a minority of healthy samples as high risk, is probably reflective some of the pitfalls associated with using citizen science data for training a disease prediction algorithm. These pitfalls include the possibility for pet owners to not be fully aware of their cat's disease status and report a cat with an undiagnosed disease (e.g., stage 1 CKD) as healthy or a cat in remission as actively suffering from a particular disease. Future iterations of the CKD training algorithm will include larger amounts of clinically recruited samples where the reported disease state of the animal comes directly from the veterinarian. This will result in improving the specificity and sensitivity of the disclosed predictive algorithm.

Study 2 demonstrated that the disclosed algorithm failed to classify cats with stage 1 CKD as being at risk for the disease. As discussed above, the inability to classify cats with stage 1 as suffering from CKD is probably associated with the fact that the healthy training cohort used for the development of the CKD prediction algorithm may have contained early stage CKD cats whose owners were not yet aware of their cat's developing renal disease. However, the disclosed algorithm was able to classify cats with stage 2 CKD as being at risk for the disease. Given the fact that most cats with CKD are formally diagnosed with the disease in stage 3, the disclosed CKD risk prediction algorithm can be a valuable pre-clinical tool used for at home disease screening by the pet owner or as part of routine veterinary visits by the veterinarian.

Using this tool has the potential to allow detection of CKD earlier and therefore aid with devising a timely and targeted treatment plan that slows disease progression. It is well known that cats diagnosed with stage 2 CKD respond well to a renal prescription diet, which in many cases is able to significantly slow down disease progression, often without further treatment.

Studies 1 and 2 focused on CKD as a case study. The results from studies 1 and 2 indicate that the disclosed computer systems, systems, algorithms and methods are capable of detecting disease states and classifying cats according to the disease state and/or a severity or grade of the disease state. It is to be understood that the disclosed methods will have a similar application and clinical utility for the detection and classification of cats with inflammatory bowel disease, diabetes mellitus, urinary calcium oxalate crystals/stones, struvite urinary crystals/stones, cystine urinary crystals/stones, and idiopathic cystitis.

The risk score generation methodology disclosed herein is based on oral microbiome compositional analysis. Other embodiments of the disclosed methods may also include incorporating predictions of the metabolic output of the oral microbiome (generated by enzymatic pathway analysis tools or metabolomics), alongside the oral microbiome compositional abundance analysis for the purpose of predictive risk of renal/urinary conditions. Other embodiments of the disclosed methods may incorporate age as a factor in the risk assessment.

### Additional Terms and Definitions

Unless defined otherwise, all technical and scientific terms used herein have the same meaning as commonly understood by one of ordinary skill in the art to which the present disclosure pertains.

Various “aspects” of the present disclosure, including systems, methods, and/or products may be illustrated with reference to one or more “embodiments,” which are exemplary in nature. As used herein, the terms “aspect” and “embodiment” may be used interchangeably. The term “embodiment” can also mean “serving as an example, instance, or illustration,” and should not necessarily be construed as preferred or advantageous over other aspects disclosed herein. In addition, reference to an “embodiment” of the present disclosure or invention is intended to provide an illustrative example without limiting the scope of the invention, which is indicated by the appended claims.

As used in this specification and the appended claims, the singular forms “a,” “an” and “the” each contemplate, include, and specifically disclose both the singular and plural referents, unless the context clearly dictates otherwise. For example, reference to a “protein” contemplates and specifically discloses one, as well as a plurality of (e.g., two or more, three or more, etc.) proteins. Similarly, use of a plural referent does not necessarily require a plurality of such referents, but contemplates, includes, specifically discloses, and/or provides support for a single, as well as a plurality of such referents, unless the context clearly dictates otherwise.

As used throughout this disclosure, the words “can” and “may” are used in a permissive sense (i.e., meaning having the potential to), rather than the mandatory sense (i.e., meaning must). Additionally, the terms “including,” “having,” “involving,” “containing,” “characterized by,” variants thereof (e.g., “includes,” “has,” and “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

The term “condition” refers to any disorder, disease, injury, or illness, as understood by those skilled in the art, that is manifested or anticipated in a patient. Manifestation of such a condition can be an early, middle, or late stage manifestation, as known in the art, including pre-condition symptoms, signs, or markers. Anticipation of such a condition can be or include the predicted, expected, envisioned, presumed, supposed, and/or speculated occurrence of the same, whether founded in scientific or medical evidence, risk assessment, or mere apprehension or trepidation.

The term “patient,” as used herein, is synonymous with the term “subject” and generally refers to any animal under the care of a medical professional, as that term is defined herein, with particular reference to (i) humans (under the care of a doctor, nurse, or medical assistant or volunteer) and (ii) non-human animals, such as non-human mammals (under the care of a veterinarian or other veterinary professional, assistant, or volunteer).

“Treating” or “treatment” as used herein covers the treatment of the disease or condition of interest in a cat, having the disease or condition of interest, and includes: (i) preventing the disease or condition from occurring in a cat, in particular, when such cat is actually starting to develop the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

For the sake of brevity, the present disclosure may recite a list or range of numerical values. It will be appreciated, however, that where such a list or range of numerical values (e.g., greater than, less than, up to, at least, and/or about a certain value, and/or between two recited values) is disclosed or recited, any specific value or range of values falling within the disclosed values or list or range of values is likewise specifically disclosed and contemplated herein.

To facilitate understanding, like references (i.e., like naming of components and/or elements) have been used, where possible, to designate like elements common to different embodiments of the present disclosure. Similarly, like components, or components with like functions, will be provided with similar reference designations, where possible. Specific language will be used herein to describe the exemplary embodiments. Nevertheless, it will be understood that no limitation of the scope of the disclosure is thereby intended. Rather, it is to be understood that the language used to describe the exemplary embodiments is illustrative only and is not to be construed as limiting the scope of the disclosure (unless such language is expressly described herein as essential).

While the detailed description is separated into sections, the section headers and contents within each section are for organizational purposes only and are not intended to be self-contained descriptions and embodiments or to limit the scope of the description or the claims.

Rather, the contents of each section within the detailed description are intended to be read and understood as a collective whole, where elements of one section may pertain to and/or inform other sections. Accordingly, embodiments specifically disclosed within one section may also relate to and/or serve as additional and/or alternative embodiments in another section having the same and/or similar products, methods, and/or terminology.

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

### Tables

TABLE 1
Table 1. Predictive microbes for chronic kidney disease (CKD), cystine urinary crystals/stones (CUCS), struvite
urinary crystals/stones (SUCS), urinary calcium oxalate crystals/stones (UCOCS), idiopathic cystitis (IC).

| CKD | CUCS | SUCS | UCOCS | IC |
|---|---|---|---|---|
| Frederiksenia | Frederiksenia | Frederiksenia | Pasteurella | Frederiksenia |
| canicola: | canicola: | canicola: | dagmatis: | canicola: |
| 123824 | 123824 | 123824 | 754 | 123824 |
| Avibacterium | Avibacterium | Glaesserella sp. | Avibacterium | Streptobacillus |
| paragallinarum: | paragallinarum: | 15-184: | paragallinarum: | moniliformis DSM |
| 728 | 728 | 2030797 | 728 | 12112: 34105 |
| Glaesserella sp. | Streptobacillus | Streptobacillus | Frederiksenia | Avibacterium |
| 15-184: | moniliformis DSM | moniliformis DSM | canicola: | paragallinarum: |
| 2030797 | 12112: 34105 | 12112: 34105 | 123824 | 728 |
| Neisseria | Haemophilus | Avibacterium | Haemophilus | Pasteurella |
| zoodegmatis: 326523 | haemolyticus: 726 | paragallinarum: 728 | haemolyticus: 726 | dagmatis: 754 |
| Pasteurella | Glaesserella sp. | Haemophilus | Glaesserella sp. | Haemophilus |
| dagmatis: 754 | 15-184: 2030797 | haemolyticus: 726 | 15-184: 2030797 | haemolyticus: 726 |
| Moraxella | Pasteurella | Pasteurella | Streptobacillus | Glaesserella sp. |
| catarrhalis | dagmatis: | dagmatis: | moniliformis DSM | 15-184: |
| BBH18: 480 | 754 | 754 | 12112: 34105 | 2030797 |
| Moraxella | Saccharomyces | Conchiformibius | Saccharomyces | Neisseria |
| bovoculi: | cerevisiae | steedae: | cerevisiae | zoodegmatis: |
| 386891 | S288C: 4932 | 153493 | S288C: 4932 | 326523 |
| Moraxella | Conchiformibius | Moraxella | Neisseria | Conchiformibius |
| cuniculi: 34061 | steedae: 153493 | cuniculi: 34061 | zoodegmatis: 326523 | steedae: 153493 |
| Streptobacillus | Neisseria | Moraxella | Conchiformibius | Moraxella |
| moniliformis DSM | canis: | catarrhalis | steedae: | cuniculi: |
| 12112: 34105 | 493 | BBH18: 480 | 153493 | 34061 |
| Conchiformibius | Neisseria | Moraxella | Moraxella | Moraxella |
| steedae: | animaloris: | bovoculi: | cuniculi: | catarrhalis |
| 153493 | 326522 | 386891 | 34061 | BBH18: 480 |
| Haemophilus | Neisseria | Saccharomyces | Moraxella | Moraxella |
| haemolyticus: | musculi: | cerevisiae | catarrhalis | bovoculi: |
| 726 | 1815583 | S288C: 4932 | BBH18: 480 | 386891 |
| Capnocytophaga | Neisseria | Neisseria | Moraxella | Histophilus |
| canimorsus | zoodegmatis: | zoodegmatis: | bovoculi: | somni 2336: |
| Cc5: 28188 | 326523 | 326523 | 386891 | 731 |
| Capnocytophaga | Neisseria | Moraxella | Neisseria | Fusobacterium |
| sp. H4358: | weaveri: | osloensis: | weaveri: | pseudoperiodonticum: |
| 1945658 | 28091 | 34062 | 28091 | 2663009 |
| Neisseria | Saccharomyces | Moraxella | Neisseria | Moraxella |
| weaveri: | eubayanus: | ovis: | animaloris: | ovis: |
| 28091 | 1080349 | 29433 | 326522 | 29433 |
| Neisseria | Pasteurella | Neisseria | Neisseria | Neisseria |
| animaloris: | multocida subsp. | animaloris: | wadsworthii: | weaveri: |
| 326522 | septica: 747 | 326522 | 607711 | 28091 |
| Moraxella | Moraxella | Neisseria | Saccharomyces | Neisseria |
| osloensis: | catarrhalis | weaveri: | eubayanus: | animaloris: |
| 34062 | BBH18: 480 | 28091 | 1080349 | 326522 |
| Moraxella | Pseudomonas | Neisseria | Neisseria | Saccharomyces |
| ovis: | sp. TKP: | musculi: | musculi: | eubayanus: |
| 29433 | 1415630 | 1815583 | 1815583 | 1080349 |
| Capnocytophaga | Fusobacterium sp. | Saccharomyces | Pseudomonas | Moraxella |
| sp. H2931: | oral taxon | eubayanus: | sp. TKP: | osloensis: |
| 1945657 | 203: 671211 | 1080349 | 1415630 | 34062 |
| Neisseria | Moraxella | Neisseria | Moraxella | Saccharomyces |
| canis: | cuniculi: | canis: | osloensis: | cerevisiae |
| 493 | 34061 | 493 | 34062 | S288C: 4932 |
| Neisseria | Moraxella | Neisseria | Neisseria | Fusobacterium |
| musculi: | bovoculi: | wadsworthii: | canis: | hwasookii ChDC |
| 1815583 | 386891 | 607711 | 493 | F300: 1583098 |
| Saccharomyces | Fusobacterium | Histophilus | Moraxella | Fusobacterium sp. |
| cerevisiae | hwasookii ChDC | somni 2336: | ovis: | oral taxon |
| S288C: 4932 | F300: 1583098 | 731 | 29433 | 203: 671211 |
| Neisseria | Neisseria | Pseudomonas | Alloprevotella | Neisseria |
| wadsworthii: | wadsworthii: | sp. TKP: | sp. E39: | wadsworthii: |
| 607711 | 607711 | 1415630 | 2133944 | 607711 |
| Lautropia | Capnocytophaga | Pasteurella | Histophilus | Fusobacterium |
| mirabilis: | canimorsus | multocida subsp. | somni | nucleatum subsp. |
| 47671 | Cc5: | septica: | 2336: | vincentii ChDC |
| | 28188 | 747 | 731 | F8: 851 |
| Saccharomyces | Fusobacterium | Capnocytophaga | Pasteurella | Fusobacterium |
| eubayanus: | pseudoperiodonticum: | canimorsus | multocida subsp. | periodonticum: |
| 1080349 | 2663009 | Cc5: 28188 | septica: 747 | 860 |
| Pasteurella | Bacillus anthracis | Cutibacterium | Psychrobacter | Neisseria |
| multocida subsp. | str. | acnes subsp. | sp. | musculi: |
| septica: | Vollum: | defendens ATCC | PRwf-1: | 1815583 |
| 747 | 1392 | 11828: 1747 | 349106 | |
| Lysobacter | Campylobacter sp. | Alloprevotella | Fusobacterium sp. | Capnocytophaga |
| oculi: | CFSAN093226: | sp. E39: | oral taxon | canimorsus |
| 2698682 | 2572065 | 2133944 | 203: 671211 | Cc5: 28188 |
| Neisseria | Vibrio sp. | Fusobacterium | Neisseria | Pasteurella |
| dentiae: | THAF191d: | sp. oral taxon | dentiae: | multocida subsp. |
| 194197 | 2661922 | 203: 671211 | 194197 | septica: 747 |
| Histophilus | Serratia | Fusobacterium | Acinetobacter | Cutibacterium |
| somni | sp. | hwasookii ChDC | johnsonii | acnes subsp. |
| 2336: | JKS000199: | F300: | XBB1: | defendens ATCC |
| 731 | 1938820 | 1583098 | 40214 | 11828: 1747 |
| Dichelobacter | Serratia sp. | Neisseria | Capnocytophaga sp. | Porphyromonas |
| nodosus | LS-1: | dentiae: | H2931: | cangingivalis: |
| VCS1703A: 870 | 2485839 | 194197 | 1945657 | 36874 |
| Wolinella | Bacteria:Spirochaetes: | Fusobacterium | Parvimonas | Capnocytophaga |
| succinogenes | Treponema | pseudoperiodonticum: | micra: | sp. |
| DSM | pallidum subsp. | 2663009 | 33033 | H4358: |
| 1740: | pertenue str. | | | 1945658 |
| 844 | SamoaD: 160 | | | |
| Neisseria | Capnocytophaga | Actinomyces | Capnocytophaga sp. | Capnocytophaga sp. |
| shayeganii: 607712 | stomatis: 1848904 | israelii: 1659 | H4358: 1945658 | H2931: 1945657 |
| Fusobacterium | Fusobacterium | Fusobacterium | Campylobacter sp. | Neisseria |
| periodonticum: | necrophorum | nucleatum | CCUG | canis: 493 |
| 860 | subsp. | subsp. vincentii | 57310: | |
| | necrophorum: 859 | ChDC F8: 851 | 2517362 | |
| Fusobacterium sp. | Porphyromonas | Capnocytophaga sp. | Porphyromonas | Parvimonas |
| oral taxon | crevioricanis: | H2931: | cangingivalis: | micra: |
| 203: 671211 | 393921 | 1945657 | 36874 | 33033 |
| Fusobacterium | Bergeyella | Capnocytophaga sp. | Porphyromonas | Alloprevotella sp. |
| pseudoperiodonticum: | cardium: | H4358: | asaccharolytica | E39: |
| 2663009 | 1585976 | 1945658 | DSM 20707: 28123 | 2133944 |
| Porphyromonas | Streptococcus | Psychrobacter | Xanthomonas | Streptococcus equi |
| asaccharolytica | pseudoporcinus: | sp. PRwf-1: | perforans | subsp. |
| DSM 20707: | 361101 | 349106 | 91-118: | zooepidemicus |
| 28123 | | | 442694 | MGCS10565: 1336 |
| Fusobacterium | Campylobacter sp. | Fusobacterium | Desulfovibrio sp. | Leptotrichia sp. |
| hwasookii ChDC | CCUG | periodonticum: | G11: | oral taxon |
| F300: 1583098 | 57310: 2517362 | 860 | 631220 | 212: 712357 |
| Streptococcus | Prevotella | Salmonella | Xanthomonas | Streptococcus |
| dysgalactiae subsp. | intermedia ATCC | enterica subsp. | translucens pv. | canis: |
| equisimilis | 25611 = DSM | salamae serovar | undulosa: | 1329 |
| RE378: | 20706: | 57: z29: | 343 | |
| 1334 | 28131 | z42: 28901 | | |
| Fusobacterium | Prevotella | Wolinella | [Arcobacter] | Porphyromonas |
| nucleatum subsp. | oris: | succinogenes | porcinus: | asaccharolytica |
| vincentii ChDC | 28135 | DSM | 1935204 | DSM 20707: |
| F8: 851 | | 1740: 844 | | 28123 |
| Corynebacterium | Chryseobacterium | Acinetobacter | Ottowia sp. | Streptococcus |
| mustelae: | gallinarum: | johnsonii | oral taxon | dysgalactiae subsp. |
| 571915 | 1324352 | XBB1: | 894: | equisimilis |
| | | 40214 | 1658672 | RE378: 1334 |
| Capnocytophaga | Cardiobacterium | Porphyromonas | Bacteroides | Capnocytophaga |
| cynodegmi: 28189 | hominis: 2718 | cangingivalis: 36874 | intestinalis: 329854 | cynodegmi: 28189 |
| Malassezia | Filifactor alocis | Leptotrichia sp. | Bacteroides | Xanthomonas sp. |
| restricta: | ATCC | oral taxon | heparinolyticus: | gxlp16: |
| 76775 | 35896: 143361 | 212: 712357 | 28113 | 2776703 |
| Psychrobacter | Gemella sp. | Porphyromonas | Bacteroides | Salmonella |
| sp. | oral taxon | asaccharolytica | caccae: | sp. |
| PRwf-1: | 928: | DSM | 47678 | S13: |
| 349106 | 1785995 | 20707: 28123 | | 2686305 |
| Salmonella enterica | Pseudopropionibacterium | Dichelobacternodosus | Bacteroides | Citrobacter |
| subsp. salamae | propionicum | VCS1703A: | uniformis: | sp. |
| serovar | F0230a: | 870 | 820 | RHB36- |
| 57: z29: | 1750 | | | C18: |
| z42: 28901 | | | | 2742627 |
| Acinetobacter | Aerococcus | Parvimonas | Pseudopropionibacterium | Citrobacter sp. |
| johnsonii | sanguinicola: | micra: | propionicum | RHBSTW |
| XBB1: 40214 | 119206 | 33033 | F0230a: 1750 | 01044: 2742678 |
| Parvimonas | Aeromonas | Prevotella fusca | Bacteroides | Salmonella sp. |
| micra: | salmonicida subsp. | JCM | cellulosilyticus: | SSDFZ54: |
| 33033 | smithia: 645 | 17724: 589436 | 246787 | 2500542 |
| Alloprevotella | Streptococcus | Streptococcus | Ottowia | [Brevibacterium] |
| sp. | anginosus subsp. | dysgalactiae | oryzae: | flavum |
| E39: | whileyi | subsp. | 2109914 | ZL-1: |
| 2133944 | MAS624: | equisimilis | | 92706 |
| | 1328 | RE378: 1334 | | |
| Porphyromonas | Campylobacter | Streptococcus | Diaphorobacter | Pseudomonas |
| cangingivalis: | showae: | equi subsp. | polyhydroxybutyrativorans: | sp. |
| 36874 | 204 | zooepidemicus | 1546149 | WCS374: |
| | | MGCS10565: 1336 | | 1495331 |
| Psychrobacter sp. | Gemella | Prevotella | Desulfomicrobium | Pseudomonas sp. |
| P11G5: | morbillorum: | enoeca: | orale DSM | J380: |
| 1699624 | 29391 | 76123 | 12838: 132132 | 2605424 |
| Lachnoanaerobaculum | Streptococcus | Actinomyces | Acidovorax ebreus | Xanthomonas |
| umeaense: | intermedius | oris: | TPSY: | perforans 91-118: |
| 617123 | JTH08: 1338 | 544580 | 721785 | 442694 |
| Enterocloster | Flavonifractor | Streptococcus | Dermabacter | Arcobacter thereius |
| clostridioformis: 1531 | plautii: 292800 | canis: 1329 | jinjuensis: 1667168 | LMG 24486: 544718 |
| Leptotrichia sp. | [Arcobacter] | Campylobacter | Ralstonia | Xanthomonas |
| oral taxon | porcinus: | sp. CCUG | mannitolilytica: | euroxanthea: |
| 212: 712357 | 1935204 | 57310: 2517362 | 105219 | 2259622 |
| Aerococcus | Comamonas | Streptococcus | Porphyromonas | Shigella |
| sanguinicola: | aquatica: | oralis subsp. | gingivalis | sonnei: |
| 119206 | 225991 | tigurinus: 1303 | W83: 837 | 624 |
| Acinetobacter | Xanthomonas | Lachnoanaerobaculum | Bacteroides | Tannerella |
| lwoffii | translucens pv. | umeaense: | caecimuris: | forsythia |
| WJ10621: 28090 | undulosa: 343 | 617123 | 1796613 | KS16: 28112 |
| Streptococcus | Prevotella | Bacillus | Bacteroides | Escherichia |
| canis: | denticola | anthracis str. | xylanisolvens: | coli str. |
| 1329 | F0289: 28129 | Vollum: 1392 | 371601 | Sanji: 562 |
| Psychrobacter sp. | Diaphorobacter | Bacillus sp. | Desulfobulbus | Desulfovibrio sp. |
| YP14: | polyhydroxybutyrativorans: | FDAARGOS_527: | oralis: | G11: |
| 2203895 | 1546149 | 2576356 | 1986146 | 631220 |
| Serratia phage | Prevotella | Streptomyces sp. | Bacteroides | Bacteroides sp. |
| Moabite: | dentalis | S1D4-14: | zoogleoformans: | A1C1: |
| 2587814 | DSM 3688: 52227 | 2594461 | 28119 | 2528203 |
| Xanthomonas sp. | Tannerella forsythia | Escherichia sp. | | Prevotella denticola |
| gxlp16: 2776703 | KS16: 28112 | SCLE84: 2725997 | | F0289: 28129 |
| Staphylococcus | Desulfovibrio sp. | Arcobacter | | Aeromonas |
| piscifermentans: | G11: | thereius LMG | | salmonicida subsp. |
| 70258 | 631220 | 24486: 5447184 | | smithia: 645 |
| Streptomyces sp. | Acidovorax | Capnocytophaga | | Bacteroides |
| PVA_94-07: | carolinensis: | stomatis: | | cellulosilyticus: |
| 1225337 | 553814 | 184890 | | 246787 |
| Enterobacter sp. | Ottowia sp. | Stenotrophomonas | | [Arcobacter] |
| RHBSTW-00593: | oral taxon | nitritireducens: | | porcinus: |
| 2742656 | 894: 1658672 | 83617 | | 1935204 |
| Methylibium sp. | Acidovorax sp. | Delftia | | Comamonas |
| T29-B: 1437443 | T1: 1858609 | tsuruhatensis: 18 | | aquatica: 225991 |
| Citrobacter sp. | Alicycliphilus | Candidatus | | Diaphorobacter |
| RHBSTW-00599: | denitrificans | Nanosynbacterlyticus: | | polyhydroxybutyrativorans: |
| 2742657 | K601: 179636 | 2093824 | | 1546149 |
| Aeromonas sp. | Dermabacter | Tannerella | | Pseudopropionibacterium |
| ASNIH7: | jinjuensis: | forsythia | | propionicum |
| 1920107 | 1667168 | KS16: 28112 | | F0230a: 1750 |
| Pseudomonas sp. | Bacteria:Spirochaetes: | Bacteroides | | Bacteroides |
| ADPe: | Treponema pedis str. | uniformis: | | intestinalis: |
| 2774873 | TA4: 409322 | 820 | | 329854 |
| Citrobacter sp. | Campylobacterrectus: | Comamonas sp. | | Bacteroides |
| RHBSTW-00570: | 203 | NLF-7-7: | | uniformis: |
| 2742655 | | 2597701 | | 820 |
| Salmonella sp. | Bacteroides | [Arcobacter] | | Comamonas sp. |
| SSDFZ69: | uniformis: | porcinus: | | NLF-7-7: |
| 2500543 | 820 | 1935204 | | 2597701 |
| Klebsiella sp. | Candidatus | Bacteroides sp. | | Bacteroides |
| MPUS7: | Nanosynbacterlyticus: | A1C1: | | fragilis |
| 2697371 | 2093824 | 2528203 | | YCH46: 817 |
| Serratia sp. | Bacteroides | Alicycliphilus | | Bacteroides |
| JKS000199: | intestinalis: | denitrificans | | caccae: |
| 1938820 | 329854 | K601: 179636 | | 47678 |
| Klebsiella sp. | Bacteria:Spirochaetes: | Cardiobacterium | | Delftia |
| WP4-W18-ESBL-05: | Treponema sp. OMZ | hominis: | | tsuruhatensis: |
| 2675713 | 838: 1539298 | 2718 | | 180282 |
| Pseudomonas sp. | Bacteria:Spirochaetes: | Bacteroides | | Ottowia sp. |
| WCS374: | Treponema sp. OMZ | fragilis | | oral taxon |
| 1495331 | 804: 120683 | YCH46: 817 | | 894: 1658672 |
| Campylobacter sp. | Melaminivora sp. | Acidovorax | | Cardiobacterium |
| CFSAN093260: | SC2-9: | carolinensis: | | hominis: |
| 2572085 | 2109913 | 553814 | | 2718 |
| Pseudomonas sp. | Ottowia | Pseudopropionibacterium | | Stenotrophomonas |
| J380: | oryzae: | propionicum | | nitritireducens: |
| 2605424 | 2109914 | F0230a: 1750 | | 83617 |
| Tessaracoccus | Bacteroides | Aeromonas sp. | | Bacteroides |
| lapidicaptus: | cellulosilyticus: | ASNIH3: | | heparinolyticus: |
| 1427523 | 246787 | 1636608 | | 28113 |
| Serratia sp. | Bacteria:Spirochaetes: | Ottowia sp. | | Lysobacter |
| LS-1: | Treponema phagedenis: | oral taxon | | oculi: |
| 2485839 | 162 | 894: 1658672 | | 2698682 |
| Xanthomonas | Campylobacter sp. | Acidovorax sp. | | Acidovorax |
| euroxanthea: | RM16192: | T1: | | carolinensis: |
| 2259622 | 1660080 | 1858609 | | 553814 |
| Bacteroides sp. | Bacteria:Spirochaetes: | Bacteroides | | Acidovorax sp. |
| HF-162: | Treponema putidum: | cellulosilyticus: | | T1: |
| 2785531 | 221027 | 246787 | | 1858609 |
| Corynebacterium | Acidovorax sp. | Xanthomonas | | Xanthomonas |
| sanguinis: | JS42: | translucens pv. | | translucens pv. |
| 2594913 | 232721 | undulosa: 343 | | undulosa: 343 |
| Bacteroides sp. | Ralstonia | Ottowia | | Melaminivora sp. |
| A1C1: | mannitolilytica: | oryzae: | | SC2-9: |
| 2528203 | 105219 | 2109914 | | 2109913 |
| Arcobacter thereius | Bacteria:Spirochaetes: | Melaminivora sp. | | Aeromonas sp. |
| LMG 24486: | Treponema denticola | SC2-9: | | ASNIH3: |
| 544718 | OTK: 158 | 2109913 | | 1636608 |
| Prevotella | Bacteroides | Ralstonia | | Stenotrophomonas |
| oris: | caccae: | mannitolilytica: | | acidaminiphila: |
| 28135 | 47678 | 105219 | | 128780 |
| Candidatus | Bacteroides | Bacteroides | | Ottowia |
| Nanosynbacter | heparinolyticus: | intestinalis: | | oryzae: |
| lyticus: 2093824 | 281131 | 329854 | | 2109914 |
| Comamonas sp. | Bacteroides | Bacteroides | | Dermabacter |
| NLF-7-7: | fragilis | heparinolyticus: | | jinjuensis: |
| 2597701 | YCH46: 817 | 28113 | | 1667168 |
| Ottowia | Porphyromonas | Bacteroides | | Porphyromonas |
| oryzae: 2109914 | gingivalis W83: 837 | caccae: 47678 | | gingivalis W83: 837 |
| Actinomyces sp. | Bacteroides | Desulfovibrio sp. | | Desulfomicrobium |
| oral taxon 171 str. | xylanisolvens: | G11: | | orale DSM |
| F0337: 706438 | 371601 | 631220 | | 12838: 132132 |
| Corynebacterium | Desulfomicrobium | Dermabacter | | Alicycliphilus |
| mycetoides: | orale DSM | jinjuensis: | | denitrificans |
| 38302 | 12838: 132132 | 1667168 | | K601: 179636 |
| Diaphorobacter | Acidovorax ebreus | Acidovorax sp. | | Bacteroides |
| polyhydroxybutyrativorans: | TPSY: | JS42: | | caecimuris: |
| 1546149 | 721785 | 232721 | | 1796613 |
| Diaphorobacter sp. | Diaphorobacter sp. | Acidovorax | | Ralstonia |
| JS3050: | JS3050: | ebreus | | mannitolilytica: |
| 2735554 | 2735554 | TPSY: 721785 | | 105219 |
| [Arcobacter] | Desulfobulbus | Bacteroides | | Desulfobulbus |
| porcinus: 1935204 | oralis: 1986146 | caecimuris: 1796613 | | oralis: 1986146 |
| Prevotella | Bacteroides | Porphyromonas | | Bacteroides |
| denticola | zoogleoformans: | gingivalis | | xylanisolvens: |
| F0289: 28129 | 28119 | W83: 837 | | 371601 |
| Tannerella | Bacteroides | Bacteroides | | Pseudomonas |
| forsythia | caecimuris: | xylanisolvens: | | denitrificans (nom. |
| KS16: 28112 | 1796613 | 371601 | | rej.): 43306 |
| Acidovorax ebreus | | Desulfomicrobium | | Acidovorax ebreus |
| TPSY: | | orale DSM | | TPSY: |
| 721785 | | 12838: 132132 | | 721785 |
| Acidovorax sp. | | Diaphorobacter | | Bacteroides |
| T1: | | sp. | | zoogleoformans: |
| 1858609 | | JS3050: 2735554 | | 28119 |
| Desulfovibrio sp. | | Desulfobulbus | | Acidovorax sp. |
| G11: 631220 | | oralis: 1986146 | | JS42: 232721 |
| Bacteroides fragilis | | Bacteroides | | Diaphorobacter sp. |
| YCH46: 817 | | zoogleoformans: 28119 | | JS3050: 2735554 |
| Actinomyces sp. | | | | |
| oral taxon | | | | |
| 169: 712116 | | | | |
| Acidovorax | | | | |
| carolinensis: 553814 | | | | |
| Porphyromonas | | | | |
| gingivalis W83: 837 | | | | |
| Acidovorax sp. | | | | |
| JS42: 232721 | | | | |
| Bacteroides | | | | |
| heparinolyticus: | | | | |
| 28113 | | | | |
| Dermabacter | | | | |
| jinjuensis: 1667168 | | | | |
| Pseudopropionibacterium | | | | |
| propionicum | | | | |
| F0230a: 1750 | | | | |
| Desulfomicrobium | | | | |
| orale DSM | | | | |
| 12838: 132132 | | | | |
| Bacteroides | | | | |
| uniformis: 820 | | | | |
| Bacteroides | | | | |
| cellulosilyticus: | | | | |
| 246787 | | | | |
| Bacteroides | | | | |
| caccae: 47678 | | | | |
| Bacteroides | | | | |
| intestinalis: 329854 | | | | |
| Desulfobulbus | | | | |
| oralis: 1986146 | | | | |
| Bacteroides | | | | |
| caecimuris: 1796613 | | | | |
| Bacteroides | | | | |
| zoogleoformans: 28119 | | | | |
| Bacteroides | | | | |
| xylanisolvens: 371601 | | | | |

TABLE 2
Table 2. Predictive microbes for Diabetes Mellitus (DM) and Inflammatory Bowel Disease (IBD).

| Diabetes, type II | IBD |
|---|---|
| Frederiksenia canicola: 123824 | Frederiksenia canicola: 123824 |
| Avibacterium paragallinarum: 728 | Streptobacillus moniliformis DSM 12112: 34105 |
| Glaesserella sp. 15-184: 2030797 | Avibacterium paragallinarum: 728 |
| Pasteurella dagmatis: 754 | Pasteurella dagmatis: 754 |
| Neisseria zoodegmatis: 326523 | Glaesserella sp. 15-184: 2030797 |
| Conchiformibius steedae: 153493 | Neisseria zoodegmatis: 326523 |
| Moraxella catarrhalis BBH18: 480 | Haemophilus haemolyticus: 726 |
| Haemophilus haemolyticus: 726 | Conchiformibius steedae: 153493 |
| Moraxella cuniculi: 34061 | Neisseria animaloris: 326522 |
| Saccharomyces cerevisiae S288C: 4932 | Neisseria weaveri: 28091 |
| Streptobacillus moniliformis DSM 12112: 34105 | Moraxella catarrhalis BBH18: 480 |
| Neisseria animaloris: 326522 | Moraxella bovoculi: 386891 |
| Neisseria weaveri: 28091 | Neisseria wadsworthii: 607711 |
| Cutibacterium acnes subsp. defendens ATCC 11828: 1747 | Moraxella cuniculi: 34061 |
| Capnocytophaga canimorsus Cc5: 28188 | Neisseria canis: 493 |
| Saccharomyces eubayanus: 1080349 | Neisseria musculi: 1815583 |
| Moraxella bovoculi: 386891 | Moraxella osloensis: 34062 |
| Neisseria musculi: 1815583 | Histophilus somni 2336: 731 |
| Neisseria canis: 493 | Saccharomyces eubayanus: 1080349 |
| Moraxella ovis: 29433 | Moraxella ovis: 29433 |
| Moraxella osloensis: 34062 | Fusobacterium pseudoperiodonticum: 2663009 |
| Capnocytophaga sp. H4358: 1945658 | Capnocytophaga canimorsus Cc5: 28188 |
| Capnocytophaga sp. H2931: 1945657 | Cutibacterium acnes subsp. defendens ATCC 11828: 1747 |
| Malassezia restricta: 76775 | Wolinella succinogenes DSM 1740: 844 |
| Histophilus somni 2336: 731 | Capnocytophaga sp. H4358: 1945658 |
| Neisseria dentiae: 194197 | Capnocytophaga sp. H2931: 1945657 |
| Neisseria wadsworthii: 607711 | Fusobacterium sp. oral taxon 203: 671211 |
| Fusobacterium pseudoperiodonticum: 2663009 | Saccharomyces cerevisiae S288C: 4932 |
| Neisseria shayeganii: 607712 | Alloprevotella sp. E39: 2133944 |
| Pasteurella multocida subsp. septica: 747 | Porphyromonas cangingivalis: 36874 |
| Streptococcus dysgalactiae subsp. equisimilis RE378: 1334 | Parvimonas micra: 33033 |
| Dichelobacter nodosus VCS1703A: 870 | Fusobacterium hwasookii ChDC F300: 1583098 |
| Capnocytophaga cynodegmi: 28189 | Pasteurella multocida subsp. septica: 747 |
| Porphyromonas cangingivalis: 36874 | Porphyromonas asaccharolytica DSM 20707: 28123 |
| Brevibacterium sp. PAMC23299: 2762330 | Malassezia restricta: 76775 |
| Serratia sp. LS-1: 2485839 | Leptotrichia sp. oral taxon 212: 712357 |
| Salmonella sp. SSDFZ69: 2500543 | Dichelobacter nodosus VCS1703A: 870 |
| Citrobacter sp. RHBSTW-00570: 2742655 | Fusobacterium nucleatum subsp. vincentii ChDC F8: 851 |
| Yersinia pestis biovar Medievalis str. Harbin 35: 632 | Prevotella fusca JCM 17724: 589436 |
| Klebsiella sp. MPUS7: 2697371 | Streptococcus equi subsp. zooepidemicus MGCS10565: 1336 |
| Klebsiella sp. WP4-W18-ESBL-05: 2675713 | Lachnoanaerobaculum umeaense: 617123 |
| Bacteroides cellulosilyticus: 246787 | Streptococcus dysgalactiae subsp. equisimilis RE378: 1334 |
| Bacteroides intestinalis: 329854 | Acinetobacter johnsonii XBB1: 40214 |
| Bacteroides caecimuris: 1796613 | Campylobacter sp. CCUG 57310: 2517362 |
| Desulfovibrio sp. G11: 631220 | Neisseria dentiae: 194197 |
| Bacteroides xylanisolvens: 371601 | Campylobacter rectus: 203 |
| Dermabacter jinjuensis: 1667168 | Fusobacterium periodonticum: 860 |
| Clostridioides difficile R20291: 1496 | Neisseria shayeganii: 607712 |
| Bacteroides zoogleoformans: 28119 | Capnocytophaga cynodegmi: 28189 |
| Acidovorax ebreus TPSY: 721785 | Streptococcus oralis subsp. tigurinus: 1303 |
| Desulfomicrobium orale DSM 12838: 132132 | Psychrobacter sp. PRwf-1: 349106 |
| Desulfobulbus oralis: 1986146 | Prevotella enoeca: 76123 |
| Porphyromonas gingivalis W83: 837 | Gemella sp. oral taxon 928: 1785995 |
| | Lautropia mirabilis: 47671 |
| | Streptococcus canis: 1329 |
| | Acinetobacter lwoffii WJ10621: 28090 |
| | Aerococcus sanguinicola: 119206 |
| | Fusobacterium necrophorum subsp. necrophorum: 859 |
| | Enterocloster clostridioformis: 1531 |
| | Filifactor alocis ATCC 35896: 143361 |
| | Porphyromonas crevioricanis: 393921 |
| | Aeromonas sp. ASNIH1: 1636606 |
| | Enterobacter sp. CRENT-193: 2051905 |
| | Streptomyces sp. ICC4: 2099584 |
| | Citrobacter sp. RHBSTW-01013: 2742677 |
| | Bacillus sp. FDAARGOS_527: 2576356 |
| | Dietzia sp. DQ12-45-1b: 912801 |
| | Citrobacter sp. RHBSTW-01044: 2742678 |
| | Streptomyces sp. S1D4-14: 2594461 |
| | Klebsiella sp. WP4-W18-ESBL-05: 2675713 |
| | Serratia sp. JKS000199: 1938820 |
| | Pseudomonas sp. EGD-AKN5: 1524461 |
| | Salmonella sp. S13: 2686305 |
| | Tessaracoccus lapidicaptus: 1427523 |
| | Xanthomonas euroxanthea: 2259622 |
| | Xanthomonas perforans 91-118: 442694 |
| | Desulfovibrio sp. G11: 631220 |
| | Actinomyces sp. oral taxon 169: 712116 |
| | Corynebacterium mycetoides: 38302 |
| | Aeromonas sp. ASNIH3: 1636608 |
| | Cardiobacterium hominis: 2718 |
| | Bacteroides heparinolyticus: 28113 |
| | Comamonas aquatica: 225991 |
| | Bacteroides sp. A1C1: 2528203 |
| | Actinomyces sp. oral taxon 171 str. F0337: 706438 |
| | Escherichia coli str. Sanji: 562 |
| | Diaphorobacter polyhydroxybutyrativorans: 1546149 |
| | Porphyromonas gingivalis W83: 837 |
| | Pseudomonas denitrificans (nom. rej.): 43306 |
| | Candidatus Nanosynbacterlyticus: 2093824 |
| | Bacteroides fragilis YCH46: 817 |
| | Ottowia sp. oral taxon 894: 1658672 |
| | Bacteroides cellulosilyticus: 246787 |
| | Comamonas sp. NLF-7-7: 2597701 |
| | Stenotrophomonas nitritireducens: 83617 |
| | Acidovorax carolinensis: 553814 |
| | Bacteroides uniformis: 820 |
| | Bacteroides caccae: 47678 |
| | Delftia tsuruhatensis: 180282 |
| | Alicycliphilus denitrificans K601: 179636 |
| | Ottowia oryzae: 2109914 |
| | Ralstonia mannitolilytica: 105219 |
| | Melaminivora sp. SC2-9: 2109913 |
| | Xanthomonas translucens pv. undulosa: 343 |
| | Pseudopropionibacterium propionicum F0230a: 1750 |
| | Bacteroides intestinalis: 329854 |
| | Acidovorax sp. T1: 1858609 |
| | Desulfomicrobium orale DSM 12838: 132132 |
| | Dermabacter jinjuensis: 1667168 |
| | Acidovorax sp. JS42: 232721 |
| | Bacteroides caecimuris: 1796613 |
| | Diaphorobacter sp. JS3050: 2735554 |
| | Desulfobulbus oralis: 1986146 |
| | Bacteroides zoogleoformans: 28119 |
| | Acidovorax ebreus TPSY: 721785 |
| | Bacteroides xylanisolvens: 371601 |

TABLE 3
Table 3. Predictive microbes alongside their taxonomic classification
for DM. Of the 53 total predictive microbes for DM (see Table 2),
approximately 9.43% are gram-positive bacteria. Note that ‘candidatus’
stands for well-characterized, but yet uncultured bacteria.

| | | |
|---|---|---|
| bacteria | proteobacteria | 60% |
| bacteria | fusobacteria | 4% |
| bacteria | firmicutes | 4% |
| bacteria | bacteroidetes | 21% |
| bacteria | spirochaetes | 0% |
| bacteria | actinobacteria | 6% |
| bacteria | candidatus | 0% |
| fungi | | 6% |
| viruses | | 0% |

TABLE 4
Table 4. Predictive microbes alongside their taxonomic classification
for IBD. Of the 116 total predictive microbes for IBD (see Table 2),
approximately 18.1% are gram-positive bacteria. Note that ‘candidatus’
stands for well-characterized, but yet uncultured bacteria.

| | | |
|---|---|---|
| bacteria | proteobacteria | 53.45% |
| bacteria | fusobacteria | 7% |
| bacteria | firmicutes | 9% |
| bacteria | bacteroidetes | 18% |
| bacteria | spirochaetes | 0% |
| bacteria | actinobacteria | 9% |
| bacteria | candidatus | 1% |
| fungi | | 3% |
| viruses | | 0% |

TABLE 5
Table 5. Predictive microbes alongside their taxonomic classification
for struvite urinary crystals/stones (SUCS). Of the 94 total
predictive microbes for SUCS (see Table 1), approximately 13.83%
are gram-positive bacteria. Note that ‘candidatus’ stands
for well-characterized, but yet uncultured bacteria.

| | | |
|---|---|---|
| bacteria | proteobacteria | 52% |
| bacteria | fusobacteria | 7% |
| bacteria | firmicutes | 9% |
| bacteria | bacteroidetes | 22% |
| bacteria | spirochaetes | 0% |
| bacteria | actinobacteria | 6% |
| bacteria | candidatus | 1% |
| fungi | | 2% |
| viruses | | 0% |

TABLE 6
Table 6. Predictive microbes alongside their taxonomic classification
for idiopathic cystitis (IC). Of the 94 total predictive
microbes for IC (see Table 1), approximately 8.51% are gram-
positive bacteria. Note that ‘candidatus’ stands for
well-characterized, but yet uncultured bacteria.

| | | |
|---|---|---|
| bacteria | proteobacteria | 61% |
| bacteria | fusobacteria | 7% |
| bacteria | firmicutes | 4% |
| bacteria | bacteroidetes | 21% |
| bacteria | spirochaetes | 0% |
| bacteria | actinobacteria | 4% |
| bacteria | candidatus | 0% |
| fungi | | 2% |
| viruses | | 0% |

TABLE 7
Table 7. Predictive microbes alongside their taxonomic classification
for cystine urinary crystals or stones (CUCS). Of the 90 total
predictive microbes for CUCS (see Table 1), approximately 12.22%
are gram-positive bacteria. Note that ‘candidatus’ stands
for well-characterized, but yet uncultured bacteria.

| | | |
|---|---|---|
| bacteria | proteobacteria | 49% |
| bacteria | fusobacteria | 6% |
| bacteria | firmicutes | 10% |
| bacteria | bacteroidetes | 22% |
| bacteria | spirochaetes | 8% |
| bacteria | actinobacteria | 2% |
| bacteria | candidatus | 1% |
| fungi | | 2% |
| viruses | | 0% |

TABLE 8
Table 8. Predictive microbes alongside their taxonomic classification
for chronic kidney disease (CKD). Of the 110 total predictive
microbes for CKD (see Table 1), approximately 14.55% are gram-
positive bacteria. Note that ‘candidatus’ stands for
well-characterized, but yet uncultured bacteria.

| | | |
|---|---|---|
| bacteria | proteobacteria | 55% |
| bacteria | fusobacteria | 6% |
| bacteria | firmicutes | 6% |
| bacteria | bacteroidetes | 20% |
| bacteria | spirochaetes | 0% |
| bacteria | actinobacteria | 8% |
| bacteria | candidatus | 1% |
| fungi | | 3% |
| viruses | | 1% |

TABLE 9
Table 9. Predictive microbes alongside their taxonomic classification
for urinary calcium oxalate crystals or stones (UCOCS). Of the
56 total predictive microbes for UCOCS (see Table 1), approximately
5.36% are gram-positive bacteria. Note that ‘candidatus’
stands for well-characterized, but yet uncultured bacteria.

| | | |
|---|---|---|
| bacteria | proteobacteria | 63% |
| bacteria | fusobacteria | 4% |
| bacteria | firmicutes | 2% |
| bacteria | bacteroidetes | 25% |
| bacteria | spirochaetes | 0% |
| bacteria | actinobacteria | 4% |
| bacteria | candidatus | 0% |
| fungi | | 4% |

TABLE 10
Table 10. The relative increased or decreased abundance
for each predictive microbe for diabetes mellitus (DM).

| | Increase/decreased relative |
|---|---|
| Microbe | abundance |
| Frederiksenia canicola: 123824 | decreased |
| Avibacterium paragallinarum: 728 | decreased |
| Glaesserella sp. 15-184: 2030797 | decreased |
| Pasteurella dagmatis: 754 | decreased |
| Neisseria zoodegmatis: 326523 | decreased |
| Conchiformibius steedae: 153493 | decreased |
| Moraxella catarrhalis BBH18: 480 | decreased |
| Haemophilus haemolyticus: 726 | decreased |
| Moraxella cuniculi: 34061 | decreased |
| Saccharomyces cerevisiae S288C: 4932 | decreased |
| Streptobacillus moniliformis DSM 12112: 34105 | decreased |
| Neisseria animaloris: 326522 | decreased |
| Neisseria weaveri: 28091 | decreased |
| Cutibacterium acnes subsp. defendens ATCC 11828: 1747 | decreased |
| Capnocytophaga canimorsus Cc5: 28188 | decreased |
| Saccharomyces eubayanus: 1080349 | decreased |
| Moraxella bovoculi: 386891 | decreased |
| Neisseria musculi: 1815583 | decreased |
| Neisseria canis: 493 | decreased |
| Moraxella ovis: 29433 | decreased |
| Moraxella osloensis: 34062 | decreased |
| Capnocytophaga sp. H4358: 1945658 | decreased |
| Capnocytophaga sp. H2931: 1945657 | decreased |
| Malassezia restricta: 76775 | decreased |
| Histophilus somni 2336: 731 | decreased |
| Neisseria dentiae: 194197 | decreased |
| Neisseria wadsworthii: 607711 | decreased |
| Fusobacterium pseudoperiodonticum: 2663009 | decreased |
| Neisseria shayeganii: 607712 | decreased |
| Pasteurella multocida subsp. septica: 747 | decreased |
| Streptococcus dysgalactiae subsp. equisimilis RE378: 1334 | decreased |
| Dichelobacter nodosus VCS1703A: 870 | decreased |
| Capnocytophaga cynodegmi: 28189 | decreased |
| Porphyromonas cangingivalis: 36874 | decreased |
| Brevibacterium sp. PAMC23299: 2762330 | increased |
| Serratia sp. LS-1: 2485839 | increased |
| Salmonella sp. SSDFZ69: 2500543 | increased |
| Citrobacter sp. RHBSTW-00570: 2742655 | increased |
| Yersinia pestis biovar Medievalis str. Harbin 35: 632 | increased |
| Klebsiella sp. MPUS7: 2697371 | increased |
| Klebsiella sp. WP4-W18-ESBL-05: 2675713 | increased |
| Bacteroides cellulosilyticus: 246787 | increased |
| Bacteroides intestinalis: 329854 | increased |
| Bacteroides caecimuris: 1796613 | increased |
| Desulfovibrio sp. G11: 631220 | increased |
| Bacteroides xylanisolvens: 371601 | increased |
| Dermabacter jinjuensis: 1667168 | increased |
| Clostridioides difficile R20291: 1496 | increased |
| Bacteroides zoogleoformans: 28119 | increased |
| Acidovorax ebreus TPSY: 721785 | increased |
| Desulfomicrobium orale DSM 12838: 132132 | increased |
| Desulfobulbus oralis: 1986146 | increased |
| Porphyromonas gingivalis W83: 837 | increased |

TABLE 11
Table 11. The relative increased or decreased abundance for
each predictive microbe for inflammatory bowel disease (IBD).

| | Increase/decreased |
|---|---|
| Microbe | relative abundance |
| Frederiksenia canicola: 123824 | decreased |
| Streptobacillus moniliformis DSM 12112: 34105 | decreased |
| Avibacterium paragallinarum: 728 | decreased |
| Pasteurella dagmatis: 754 | decreased |
| Glaesserella sp. 15-184: 2030797 | decreased |
| Neisseria zoodegmatis: 326523 | decreased |
| Haemophilus haemolyticus: 726 | decreased |
| Conchiformibius steedae: 153493 | decreased |
| Neisseria animaloris: 326522 | decreased |
| Neisseria weaveri: 28091 | decreased |
| Moraxella catarrhalis BBH18: 480 | decreased |
| Moraxella bovoculi: 386891 | decreased |
| Neisseria wadsworthii: 607711 | decreased |
| Moraxella cuniculi: 34061 | decreased |
| Neisseria canis: 493 | decreased |
| Neisseria musculi: 1815583 | decreased |
| Moraxella osloensis: 34062 | decreased |
| Histophilus somni 2336: 731 | decreased |
| Saccharomyces eubayanus: 1080349 | decreased |
| Moraxella ovis: 29433 | decreased |
| Fusobacterium pseudoperiodonticum: 2663009 | decreased |
| Capnocytophaga canimorsus Cc5: 28188 | decreased |
| Cutibacterium acnes subsp. defendens ATCC 11828: 1747 | decreased |
| Wolinella succinogenes DSM 1740: 844 | decreased |
| Capnocytophaga sp. H4358: 1945658 | decreased |
| Capnocytophaga sp. H2931: 1945657 | decreased |
| Fusobacterium sp. oral taxon 203: 671211 | decreased |
| Saccharomyces cerevisiae S288C: 4932 | decreased |
| Alloprevotella sp. E39: 2133944 | decreased |
| Porphyromonas cangingivalis: 36874 | decreased |
| Parvimonas micra: 33033 | decreased |
| Fusobacterium hwasookii ChDC F300: 1583098 | decreased |
| Pasteurella multocida subsp. septica: 747 | decreased |
| Porphyromonas asaccharolytica DSM 20707: 28123 | decreased |
| Malassezia restricta: 76775 | decreased |
| Leptotrichia sp. oral taxon 212: 712357 | decreased |
| Dichelobacter nodosus VCS1703A: 870 | decreased |
| Fusobacterium nucleatum subsp. vincentii ChDC F8: 851 | decreased |
| Prevotella fusca JCM 17724: 589436 | decreased |
| Streptococcus equi subsp. zooepidemicus | decreased |
| MGCS10565: 1336 | |
| Lachnoanaerobaculum umeaense: 617123 | decreased |
| Streptococcus dysgalactiae subsp. equisimilis RE378: 1334 | decreased |
| Acinetobacter johnsonii XBB1: 40214 | decreased |
| Campylobacter sp. CCUG 57310: 2517362 | decreased |
| Neisseria dentiae: 194197 | decreased |
| Campylobacter rectus: 203 | decreased |
| Fusobacterium periodonticum: 860 | decreased |
| Neisseria shayeganii: 607712 | decreased |
| Capnocytophaga cynodegmi: 28189 | decreased |
| Streptococcus oralis subsp. tigurinus: 1303 | decreased |
| Psychrobacter sp. PRwf-1: 349106 | decreased |
| Prevotella enoeca: 76123 | decreased |
| Gemella sp. oral taxon 928: 1785995 | decreased |
| Lautropia mirabilis: 47671 | decreased |
| Streptococcus canis: 1329 | decreased |
| Acinetobacter lwoffii WJ10621: 28090 | decreased |
| Aerococcus sanguinicola: 119206 | decreased |
| Fusobacterium necrophorum subsp. necrophorum: 859 | decreased |
| Enterocloster clostridioformis: 1531 | decreased |
| Filifactor alocis ATCC 35896: 143361 | decreased |
| Porphyromonas crevioricanis: 393921 | decreased |
| Aeromonas sp. ASNIH1: 1636606 | decreased |
| Enterobacter sp. CRENT-193: 2051905 | increased |
| Streptomyces sp. ICC4: 2099584 | increased |
| Citrobacter sp. RHBSTW-01013: 2742677 | increased |
| Bacillus sp. FDAARGOS_527: 2576356 | increased |
| Dietzia sp. DQ12-45-1b: 912801 | increased |
| Citrobacter sp. RHBSTW-01044: 2742678 | increased |
| Streptomyces sp. S1D4-14: 2594461 | increased |
| Klebsiella sp. WP4-W18-ESBL-05: 2675713 | increased |
| Serratia sp. JKS000199: 1938820 | increased |
| Pseudomonas sp. EGD-AKN5: 1524461 | increased |
| Salmonella sp. S13: 2686305 | increased |
| Tessaracoccus lapidicaptus: 1427523 | increased |
| Xanthomonas euroxanthea: 2259622 | increased |
| Xanthomonas perforans 91-118: 442694 | increased |
| Desulfovibrio sp. G11: 631220 | increased |
| Actinomyces sp. oral taxon 169: 712116 | increased |
| Corynebacterium mycetoides: 38302 | increased |
| Aeromonas sp. ASNIH3: 1636608 | increased |
| Cardiobacterium hominis: 2718 | increased |
| Bacteroides heparinolyticus: 28113 | increased |
| Comamonas aquatica: 225991 | increased |
| Bacteroides sp. A1C1: 2528203 | increased |
| Actinomyces sp. oral taxon 171 str. F0337: 706438 | increased |
| Escherichia coli str. Sanji: 562 | increased |
| Diaphorobacter polyhydroxybutyrativorans: 1546149 | increased |
| Porphyromonas gingivalis W83: 837 | increased |
| Pseudomonas denitrificans (nom. rej.): 43306 | increased |
| Candidatus Nanosynbacter lyticus: 2093824 | increased |
| Bacteroides fragilis YCH46: 817 | increased |
| Ottowia sp. oral taxon 894: 1658672 | increased |
| Bacteroides cellulosilyticus: 246787 | increased |
| Comamonas sp. NLF-7-7: 2597701 | increased |
| Stenotrophomonas nitritireducens: 83617 | increased |
| Acidovorax carolinensis: 553814 | increased |
| Bacteroides uniformis: 820 | increased |
| Bacteroides caccae: 47678 | increased |
| Delftia tsuruhatensis: 180282 | increased |
| Alicycliphilus denitrificans K601: 179636 | increased |
| Ottowia oryzae: 2109914 | increased |
| Ralstonia mannitolilytica: 105219 | increased |
| Melaminivora sp. SC2-9: 2109913 | increased |
| Xanthomonas translucens pv. undulosa: 343 | increased |
| Pseudopropionibacterium propionicum F0230a: 1750 | increased |
| Bacteroides intestinalis: 329854 | increased |
| Acidovorax sp. T1: 1858609 | increased |
| Desulfomicrobium orale DSM 12838: 132132 | increased |
| Dermabacter jinjuensis: 1667168 | increased |
| Acidovorax sp. JS42: 232721 | increased |
| Bacteroides caecimuris: 1796613 | increased |
| Diaphorobacter sp. JS3050: 2735554 | increased |
| Desulfobulbus oralis: 1986146 | increased |
| Bacteroides zoogleoformans: 28119 | increased |
| Acidovorax ebreus TPSY: 721785 | increased |
| Bacteroides xylanisolvens: 371601 | increased |

TABLE 12
Table 12. The relative increased or decreased abundance for
each predictive microbe for chronic kidney disease (CKD).

| | Increase/decreased relative |
|---|---|
| Microbe | abundance |
| Frederiksenia canicola: 123824 | decreased |
| Avibacterium paragallinarum: 728 | decreased |
| Glaesserella sp. 15-184: 2030797 | decreased |
| Neisseria zoodegmatis: 326523 | decreased |
| Pasteurella dagmatis: 754 | decreased |
| Moraxella catarrhalis BBH18: 480 | decreased |
| Moraxella bovoculi: 386891 | decreased |
| Moraxella cuniculi: 34061 | decreased |
| Streptobacillus moniliformis DSM 12112: 34105 | decreased |
| Conchiformibius steedae: 153493 | decreased |
| Haemophilus haemolyticus: 726 | decreased |
| Capnocytophaga canimorsus Cc5: 28188 | decreased |
| Capnocytophaga sp. H4358: 1945658 | decreased |
| Neisseria weaveri: 28091 | decreased |
| Neisseria animaloris: 326522 | decreased |
| Moraxella osloensis: 34062 | decreased |
| Moraxella ovis: 29433 | decreased |
| Capnocytophaga sp. H2931: 1945657 | decreased |
| Neisseria canis: 493 | decreased |
| Neisseria musculi: 1815583 | decreased |
| Saccharomyces cerevisiae S288C: 4932 | decreased |
| Neisseria wadsworthii: 607711 | decreased |
| Lautropia mirabilis: 47671 | decreased |
| Saccharomyces eubayanus: 1080349 | decreased |
| Pasteurella multocida subsp. septica: 747 | decreased |
| Lysobacter oculi: 2698682 | decreased |
| Neisseria dentiae: 194197 | decreased |
| Histophilus somni 2336: 731 | decreased |
| Dichelobacter nodosus VCS1703A: 870 | decreased |
| Wolinella succinogenes DSM 1740: 844 | decreased |
| Neisseria shayeganii: 607712 | decreased |
| Fusobacterium periodonticum: 860 | decreased |
| Fusobacterium sp. oral taxon 203: 671211 | decreased |
| Fusobacterium pseudoperiodonticum: 2663009 | decreased |
| Porphyromonas asaccharolytica DSM 20707: 28123 | decreased |
| Fusobacterium hwasookii ChDC F300: 1583098 | decreased |
| Streptococcus dysgalactiae subsp. equisimilis RE378: 1334 | decreased |
| Fusobacterium nucleatum subsp. vincentii ChDC F8: 851 | decreased |
| Corynebacterium mustelae: 571915 | decreased |
| Capnocytophaga cynodegmi: 28189 | decreased |
| Malassezia restricta: 76775 | decreased |
| Psychrobacter sp. PRwf-1: 349106 | decreased |
| Salmonella enterica subsp. salamae serovar | decreased |
| 57: z29: z42: 28901 | |
| Acinetobacter johnsonii XBB1: 40214 | decreased |
| Parvimonas micra: 33033 | decreased |
| Alloprevotella sp. E39: 2133944 | decreased |
| Porphyromonas cangingivalis: 36874 | decreased |
| Psychrobacter sp. P11G5: 1699624 | decreased |
| Lachnoanaerobaculum umeaense: 617123 | decreased |
| Enterocloster clostridioformis: 1531 | decreased |
| Leptotrichia sp. oral taxon 212: 712357 | decreased |
| Aerococcus sanguinicola: 119206 | decreased |
| Acinetobacter lwoffii WJ10621: 28090 | decreased |
| Streptococcus canis: 1329 | decreased |
| Psychrobacter sp. YP14: 2203895 | decreased |
| Serratia phage Moabite: 2587814 | increased |
| Xanthomonas sp. gxlp16: 2776703 | increased |
| Staphylococcus piscifermentans: 70258 | increased |
| Streptomyces sp. PVA_94-07: 1225337 | increased |
| Enterobacter sp. RHBSTW-00593: 2742656 | increased |
| Methylibium sp. T29-B: 1437443 | increased |
| Citrobacter sp. RHBSTW-00599: 2742657 | increased |
| Aeromonas sp. ASNIH7: 1920107 | increased |
| Pseudomonas sp. ADPe: 2774873 | increased |
| Citrobacter sp. RHBSTW-00570: 2742655 | increased |
| Salmonella sp. SSDFZ69: 2500543 | increased |
| Klebsiella sp. MPUS7: 2697371 | increased |
| Serratia sp. JKS000199: 1938820 | increased |
| Klebsiella sp. WP4-W18-ESBL-05: 2675713 | increased |
| Pseudomonas sp. WCS374: 1495331 | increased |
| Campylobacter sp. CFSAN093260: 2572085 | increased |
| Pseudomonas sp. J380: 2605424 | increased |
| Tessaracoccus lapidicaptus: 1427523 | increased |
| Serratia sp. LS-1: 2485839 | increased |
| Xanthomonas euroxanthea: 2259622 | increased |
| Bacteroides sp. HF-162: 2785531 | increased |
| Corynebacterium sanguinis: 2594913 | increased |
| Bacteroides sp. A1C1: 2528203 | increased |
| Arcobacter thereius LMG 24486: 544718 | increased |
| Prevotella oris: 28135 | increased |
| Candidatus Nanosynbacter lyticus: 2093824 | increased |
| Comamonas sp. NLF-7-7: 2597701 | increased |
| Ottowia oryzae: 2109914 | increased |
| Actinomyces sp. oral taxon 171 str. F0337: 706438 | increased |
| Corynebacterium mycetoides: 38302 | increased |
| Diaphorobacter polyhydroxybutyrativorans: 1546149 | increased |
| Diaphorobacter sp. JS3050: 2735554 | increased |
| [Arcobacter] porcinus: 1935204 | increased |
| Prevotella denticola F0289: 28129 | increased |
| Tannerella forsythia KS16: 28112 | increased |
| Acidovorax ebreus TPSY: 721785 | increased |
| Acidovorax sp. T1: 1858609 | increased |
| Desulfovibrio sp. G11: 631220 | increased |
| Bacteroides fragilis YCH46: 817 | increased |
| Actinomyces sp. oral taxon 169: 712116 | increased |
| Acidovorax carolinensis: 553814 | increased |
| Porphyromonas gingivalis W83: 837 | increased |
| Acidovorax sp. JS42: 232721 | increased |
| Bacteroides heparinolyticus: 28113 | increased |
| Dermabacter jinjuensis: 1667168 | increased |
| Pseudopropionibacterium propionicum F0230a: 1750 | increased |
| Desulfomicrobium orale DSM 12838: 132132 | increased |
| Bacteroides uniformis: 820 | increased |
| Bacteroides cellulosilyticus: 246787 | increased |
| Bacteroides caccae: 47678 | increased |
| Bacteroides intestinalis: 329854 | increased |
| Desulfobulbus oralis: 1986146 | increased |
| Bacteroides caecimuris: 1796613 | increased |
| Bacteroides zoogleoformans: 28119 | increased |
| Bacteroides xylanisolvens: 371601 | increased |

TABLE 13
Table 13. The relative increased or decreased abundance for each
predictive microbe for struvite urinary crystals/stones (SUCS).

| | Increase/decreased |
|---|---|
| Microbe | relative abundance |
| Frederiksenia canicola: 123824 | decreased |
| Glaesserella sp. 15-184: 2030797 | decreased |
| Streptobacillus moniliformis DSM 12112: 34105 | decreased |
| Avibacterium paragallinarum: 728 | decreased |
| Haemophilus haemolyticus: 726 | decreased |
| Pasteurella dagmatis: 754 | decreased |
| Conchiformibius steedae: 153493 | decreased |
| Moraxella cuniculi: 34061 | decreased |
| Moraxella catarrhalis BBH18: 480 | decreased |
| Moraxella bovoculi: 386891 | decreased |
| Saccharomyces cerevisiae S288C: 4932 | decreased |
| Neisseria zoodegmatis: 326523 | decreased |
| Moraxella osloensis: 34062 | decreased |
| Moraxella ovis: 29433 | decreased |
| Neisseria animaloris: 326522 | decreased |
| Neisseria weaveri: 28091 | decreased |
| Neisseria musculi: 1815583 | decreased |
| Saccharomyces eubayanus: 1080349 | decreased |
| Neisseria canis: 493 | decreased |
| Neisseria wadsworthii: 607711 | decreased |
| Histophilus somni 2336: 731 | decreased |
| Pseudomonas sp. TKP: 1415630 | decreased |
| Pasteurella multocida subsp. septica: 747 | decreased |
| Capnocytophaga canimorsus Cc5: 28188 | decreased |
| Cutibacterium acnes subsp. defendens ATCC 11828: 1747 | decreased |
| Alloprevotella sp. E39: 2133944 | decreased |
| Fusobacterium sp. oral taxon 203: 671211 | decreased |
| Fusobacterium hwasookii ChDC F300: 1583098 | decreased |
| Neisseria dentiae: 194197 | decreased |
| Fusobacterium pseudoperiodonticum: 2663009 | decreased |
| Actinomyces israelii: 1659 | decreased |
| Fusobacterium nucleatum subsp. vincentii ChDC F8: 851 | decreased |
| Capnocytophaga sp. H2931: 1945657 | decreased |
| Capnocytophaga sp. H4358: 1945658 | decreased |
| Psychrobacter sp. PRwf-1: 349106 | decreased |
| Fusobacterium periodonticum: 860 | decreased |
| Salmonella enterica subsp. salamae serovar | decreased |
| 57: z29: z42: 28901 | |
| Wolinella succinogenes DSM 1740: 844 | decreased |
| Acinetobacter johnsonii XBB1: 40214 | decreased |
| Porphyromonas cangingivalis: 36874 | decreased |
| Leptotrichia sp. oral taxon 212: 712357 | decreased |
| Porphyromonas asaccharolytica DSM 20707: 28123 | decreased |
| Dichelobacter nodosus VCS1703A: 870 | decreased |
| Parvimonas micra: 33033 | decreased |
| Prevotella fusca JCM 17724: 589436 | decreased |
| Streptococcus dysgalactiae subsp. equisimilis RE378: 1334 | decreased |
| Streptococcus equi subsp. zooepidemicus MGCS10565: 1336 | decreased |
| Prevotella enoeca: 76123 | decreased |
| Actinomyces oris: 544580 | decreased |
| Streptococcus canis: 1329 | decreased |
| Campylobacter sp. CCUG 57310: 2517362 | decreased |
| Streptococcus oralis subsp. tigurinus: 1303 | decreased |
| Lachnoanaerobaculum umeaense: 617123 | decreased |
| Bacillus anthracis str. Vollum: 1392 | decreased |
| Bacillus sp. FDAARGOS_527: 2576356 | increased |
| Streptomyces sp. S1D4-14: 2594461 | increased |
| Escherichia sp. SCLE84: 2725997 | increased |
| Arcobacter thereius LMG 24486: 544718 | increased |
| Capnocytophaga stomatis: 1848904 | increased |
| Stenotrophomonas nitritireducens: 83617 | increased |
| Delftia tsuruhatensis: 180282 | increased |
| Candidatus Nanosynbacter lyticus: 2093824 | increased |
| Tannerella forsythia KS16: 28112 | increased |
| Bacteroides uniformis: 820 | increased |
| Comamonas sp. NLF-7-7: 2597701 | increased |
| [Arcobacter] porcinus: 1935204 | increased |
| Bacteroides sp. A1C1: 2528203 | increased |
| Alicycliphilus denitrificans K601: 179636 | increased |
| Cardiobacterium hominis: 2718 | increased |
| Bacteroides fragilis YCH46: 817 | increased |
| Acidovorax carolinensis: 553814 | increased |
| Pseudopropionibacterium propionicum F0230a: 1750 | increased |
| Aeromonas sp. ASNIH3: 1636608 | increased |
| Ottowia sp. oral taxon 894: 1658672 | increased |
| Acidovorax sp. T1: 1858609 | increased |
| Bacteroides cellulosilyticus: 246787 | increased |
| Xanthomonas translucens pv. undulosa: 343 | increased |
| Ottowia oryzae: 2109914 | increased |
| Melaminivora sp. SC2-9: 2109913 | increased |
| Ralstonia mannitolilytica: 105219 | increased |
| Bacteroides intestinalis: 329854 | increased |
| Bacteroides heparinolyticus: 28113 | increased |
| Bacteroides caccae: 47678 | increased |
| Desulfovibrio sp. G11: 631220 | increased |
| Dermabacter jinjuensis: 1667168 | increased |
| Acidovorax sp. JS42: 232721 | increased |
| Acidovorax ebreus TPSY: 721785 | increased |
| Bacteroides caecimuris: 1796613 | increased |
| Porphyromonas gingivalis W83: 837 | increased |
| Bacteroides xylanisolvens: 371601 | increased |
| Desulfomicrobium orale DSM 12838: 132132 | increased |
| Diaphorobacter sp. JS3050: 2735554 | increased |
| Desulfobulbus oralis: 1986146 | increased |
| Bacteroides zoogleoformans: 28119 | increased |

TABLE 14
Table 14. The relative increased or decreased abundance for each predictive
microbe for urinary calcium oxalate crystals or stones (UCOCS).

| | Increase/decreased relative |
|---|---|
| Microbe | abundance |
| Pasteurella dagmatis: 754 | decreased |
| Avibacterium paragallinarum: 728 | decreased |
| Frederiksenia canicola: 123824 | decreased |
| Haemophilus haemolyticus: 726 | decreased |
| Glaesserella sp. 15-184: 2030797 | decreased |
| Streptobacillus moniliformis DSM 12112: 34105 | decreased |
| Saccharomyces cerevisiae S288C: 4932 | decreased |
| Neisseria zoodegmatis: 326523 | decreased |
| Conchiformibius steedae: 153493 | decreased |
| Moraxella cuniculi: 34061 | decreased |
| Moraxella catarrhalis BBH18: 480 | decreased |
| Moraxella bovoculi: 386891 | decreased |
| Neisseria weaveri: 28091 | decreased |
| Neisseria animaloris: 326522 | decreased |
| Neisseria wadsworthii: 607711 | decreased |
| Saccharomyces eubayanus: 1080349 | decreased |
| Neisseria musculi: 1815583 | decreased |
| Pseudomonas sp. TKP: 1415630 | decreased |
| Moraxella osloensis: 34062 | decreased |
| Neisseria canis: 493 | decreased |
| Moraxella ovis: 29433 | decreased |
| Alloprevotella sp. E39: 2133944 | decreased |
| Histophilus somni 2336: 731 | decreased |
| Pasteurella multocida subsp. septica: 747 | decreased |
| Psychrobacter sp. PRwf-1: 349106 | decreased |
| Fusobacterium sp. oral taxon 203: 671211 | decreased |
| Neisseria dentiae: 194197 | decreased |
| Acinetobacter johnsonii XBB1: 40214 | decreased |
| Capnocytophaga sp. H2931: 1945657 | decreased |
| Parvimonas micra: 33033 | decreased |
| Capnocytophaga sp. H4358: 1945658 | decreased |
| Campylobacter sp. CCUG 57310: 2517362 | decreased |
| Porphyromonas cangingivalis: 36874 | decreased |
| Porphyromonas asaccharolytica DSM 20707: 28123 | decreased |
| Xanthomonas perforans 91-118: 442694 | increased |
| Desulfovibrio sp. G11: 631220 | increased |
| Xanthomonas translucens pv. undulosa: 343 | increased |
| [Arcobacter] porcinus: 1935204 | increased |
| Ottowia sp. oral taxon 894: 1658672 | increased |
| Bacteroides intestinalis: 329854 | increased |
| Bacteroides heparinolyticus: 28113 | increased |
| Bacteroides caccae: 47678 | increased |
| Bacteroides uniformis: 820 | increased |
| Pseudopropionibacterium propionicum F0230a: 1750 | increased |
| Bacteroides cellulosilyticus: 246787 | increased |
| Ottowia oryzae: 2109914 | increased |
| Diaphorobacter polyhydroxybutyrativorans: 1546149 | increased |
| Desulfomicrobium orale DSM 12838: 132132 | increased |
| Acidovorax ebreus TPSY: 721785 | increased |
| Dermabacter jinjuensis: 1667168 | increased |
| Ralstonia mannitolilytica: 105219 | increased |
| Porphyromonas gingivalis W83: 837 | increased |
| Bacteroides caecimuris: 1796613 | increased |
| Bacteroides xylanisolvens: 371601 | increased |
| Desulfobulbus oralis: 1986146 | increased |
| Bacteroides zoogleoformans: 28119 | increased |

TABLE 15
Table 15. The relative increased or decreased abundance for each
predictive microbe for cystine urinary crystals or stones (CUCS).

| | Increase/decreased |
|---|---|
| CUCS | relative abundance |
| Frederiksenia canicola: 123824 | decreased |
| Avibacterium paragallinarum: 728 | decreased |
| Streptobacillus moniliformis DSM 12112: 34105 | decreased |
| Haemophilus haemolyticus: 726 | decreased |
| Glaesserella sp. 15-184: 2030797 | decreased |
| Pasteurella dagmatis: 754 | decreased |
| Saccharomyces cerevisiae S288C: 4932 | decreased |
| Conchiformibius steedae: 153493 | decreased |
| Neisseria canis: 493 | decreased |
| Neisseria animaloris: 326522 | decreased |
| Neisseria musculi: 1815583 | decreased |
| Neisseria zoodegmatis: 326523 | decreased |
| Neisseria weaveri: 28091 | decreased |
| Saccharomyces eubayanus: 1080349 | decreased |
| Pasteurella multocida subsp. septica: 747 | decreased |
| Moraxella catarrhalis BBH18: 480 | decreased |
| Pseudomonas sp. TKP: 1415630 | decreased |
| Fusobacteriumsp. oral taxon 203: 671211 | decreased |
| Moraxella cuniculi: 34061 | decreased |
| Moraxella bovoculi: 386891 | decreased |
| Fusobacterium hwasookii ChDC F300: 1583098 | decreased |
| Neisseria wadsworthii: 607711 | decreased |
| Capnocytophaga canimorsus Cc5: 28188 | decreased |
| Fusobacterium pseudoperiodonticum: 2663009 | decreased |
| Bacillus anthracis str. Vollum: 1392 | decreased |
| Campylobacter sp. CFSAN093226: 2572065 | decreased |
| Vibrio sp. THAF191d: 2661922 | decreased |
| Serratia sp. JKS000199: 1938820 | decreased |
| Serratia sp. LS-1: 2485839 | decreased |
| Bacteria: Spirochaetes: Treponema pallidum subsp. pertenue str. | increased |
| SamoaD: 160 | |
| Capnocytophaga stomatis: 1848904 | increased |
| Fusobacterium necrophorum subsp. necrophorum: 859 | increased |
| Porphyromonas crevioricanis: 393921 | increased |
| Bergeyella cardium: 1585976 | increased |
| Streptococcus pseudoporcinus: 361101 | increased |
| Campylobacter sp. CCUG 57310: 2517362 | increased |
| Prevotella intermedia ATCC 25611 = DSM 20706: 28131 | increased |
| Prevotella oris: 28135 | increased |
| Chryseobacterium gallinarum: 1324352 | increased |
| Cardiobacterium hominis: 2718 | increased |
| Filifactor alocis ATCC 35896: 143361 | increased |
| Gemella sp. oral taxon 928: 1785995 | increased |
| Pseudopropionibacterium propionicum F0230a: 1750 | increased |
| Aerococcus sanguinicola: 119206 | increased |
| Aeromonas salmonicida subsp. smithia: 645 | increased |
| Streptococcus anginosus subsp. whileyi MAS624: 1328 | increased |
| Campylobacter showae: 204 | increased |
| Gemella morbillorum: 29391 | increased |
| Streptococcus intermedius JTH08: 1338 | increased |
| Flavonifractor plautii: 292800 | increased |
| [Arcobacter] porcinus: 1935204 | increased |
| Comamonas aquatica: 225991 | increased |
| Xanthomonas translucens pv. undulosa: 343 | increased |
| Prevotella denticola F0289: 28129 | increased |
| Diaphorobacter polyhydroxybutyrativorans: 1546149 | increased |
| Prevotella dentalis DSM 3688: 52227 | increased |
| Tannerella forsythia KS16: 28112 | increased |
| Desulfovibrio sp. G11: 631220 | increased |
| Acidovorax carolinensis: 553814 | increased |
| Ottowia sp. oral taxon 894: 1658672 | increased |
| Acidovorax sp. T1: 1858609 | increased |
| Alicycliphilus denitrificans K601: 179636 | increased |
| Dermabacter jinjuensis: 1667168 | increased |
| Bacteria: Spirochaetes: Treponema pedis str. T A4: 409322 | increased |
| Campylobacter rectus: 203 | increased |
| Bacteroides uniformis: 820 | increased |
| Candidatus Nanosynbacter lyticus: 2093824 | increased |
| Bacteroides intestinalis: 329854 | increased |
| Bacteria: Spirochaetes: Treponema sp. OMZ 838: 1539298 | increased |
| Bacteria: Spirochaetes: Treponema sp. OMZ 804: 120683 | increased |
| Melaminivora sp. SC2-9: 2109913 | increased |
| Ottowia oryzae: 2109914 | increased |
| Bacteroides cellulosilyticus: 246787 | increased |
| Bacteria: Spirochaetes: Treponema phagedenis: 162 | increased |
| Campylobacter sp. RM16192: 1660080 | increased |
| Bacteria: Spirochaetes: Treponema putidum: 221027 | increased |
| Acidovorax sp. JS42: 232721 | increased |
| Ralstonia mannitolilytica: 105219 | increased |
| Bacteria: Spirochaetes: Treponema denticola OTK: 158 | increased |
| Bacteroides caccae: 47678 | increased |
| Bacteroides heparinolyticus: 28113 | increased |
| Bacteroides fragilis YCH46: 817 | increased |
| Porphyromonas gingivalis W83: 837 | increased |
| Bacteroides xylanisolvens: 371601 | increased |
| Desulfomicrobium orale DSM 12838: 132132 | increased |
| Acidovorax ebreus TPSY: 721785 | increased |
| Diaphorobacter sp. JS3050: 2735554 | increased |
| Desulfobulbus oralis: 1986146 | increased |
| Bacteroides zoogleoformans: 28119 | increased |
| Bacteroides caecimuris: 1796613 | increased |

TABLE 16
Table 16. The relative increased or decreased abundance
for each predictive microbe for idiopathic cystitis (IC).

| | Increase/decreased relative |
|---|---|
| Microbe | abundance |
| Frederiksenia canicola: 123824 | decreased |
| Streptobacillus moniliformis DSM 12112: 34105 | decreased |
| Avibacterium paragallinarum: 728 | decreased |
| Pasteurella dagmatis: 754 | decreased |
| Haemophilus haemolyticus: 726 | decreased |
| Glaesserella sp. 15-184: 2030797 | decreased |
| Neisseria zoodegmatis: 326523 | decreased |
| Conchiformibius steedae: 153493 | decreased |
| Moraxella cuniculi: 34061 | decreased |
| Moraxella catarrhalis BBH18: 480 | decreased |
| Moraxella bovoculi: 386891 | decreased |
| Histophilus somni 2336: 731 | decreased |
| Fusobacterium pseudoperiodonticum: 2663009 | decreased |
| Moraxella ovis: 29433 | decreased |
| Neisseria weaveri: 28091 | decreased |
| Neisseria animaloris: 326522 | decreased |
| Saccharomyces eubayanus: 1080349 | decreased |
| Moraxella osloensis: 34062 | decreased |
| Saccharomyces cerevisiae S288C: 4932 | decreased |
| Fusobacterium hwasookii ChDC F300: 1583098 | decreased |
| Fusobacterium sp. oral taxon 203: 671211 | decreased |
| Neisseria wadsworthii: 607711 | decreased |
| Fusobacterium nucleatum subsp. vincentii ChDC F8: 851 | decreased |
| Fusobacterium periodonticum: 860 | decreased |
| Neisseria musculi: 1815583 | decreased |
| Capnocytophaga canimorsus Cc5: 28188 | decreased |
| Pasteurella multocida subsp. septica: 747 | decreased |
| Cutibacterium acnes subsp. defendens ATCC 11828: 1747 | decreased |
| Porphyromonas cangingivalis: 36874 | decreased |
| Capnocytophaga sp. H4358: 1945658 | decreased |
| Capnocytophaga sp. H2931: 1945657 | decreased |
| Neisseria canis: 493 | decreased |
| Parvimonas micra: 33033 | decreased |
| Alloprevotella sp. E39: 2133944 | decreased |
| Streptococcus equi subsp. zooepidemicus MGCS10565: 1336 | decreased |
| Leptotrichia sp. oral taxon 212: 712357 | decreased |
| Streptococcus canis: 1329 | decreased |
| Porphyromonas asaccharolytica DSM 20707: 28123 | decreased |
| Streptococcus dysgalactiae subsp. equisimilis RE378: 1334 | decreased |
| Capnocytophaga cynodegmi: 28189 | decreased |
| Xanthomonas sp. gxlp16: 2776703 | increased |
| Salmonella sp. S13: 2686305 | increased |
| Citrobacter sp. RHB36-C18: 2742627 | increased |
| Citrobacter sp. RHBSTW-01044: 2742678 | increased |
| Salmonella sp. SSDFZ54: 2500542 | increased |
| [Brevibacterium] flavum ZL-1: 92706 | increased |
| Pseudomonas sp. WCS374: 1495331 | increased |
| Pseudomonas sp. J380: 2605424 | increased |
| Xanthomonas perforans 91-118: 442694 | increased |
| Arcobacter thereius LMG 24486: 544718 | increased |
| Xanthomonas euroxanthea: 2259622 | increased |
| Shigella sonnei: 624 | increased |
| Tannerella forsythia KS16: 28112 | increased |
| Escherichia coli str. Sanji: 562 | increased |
| Desulfovibrio sp. G11: 631220 | increased |
| Bacteroides sp. A1C1: 2528203 | increased |
| Prevotella denticola F0289: 28129 | increased |
| Aeromonas salmonicida subsp. smithia: 645 | increased |
| Bacteroides cellulosilyticus: 246787 | increased |
| [Arcobacter] porcinus: 1935204 | increased |
| Comamonas aquatica: 225991 | increased |
| Diaphorobacter polyhydroxybutyrativorans: 1546149 | increased |
| Pseudopropionibacterium propionicum F0230a: 1750 | increased |
| Bacteroides intestinalis: 329854 | increased |
| Bacteroides uniformis: 820 | increased |
| Comamonas sp. NLF-7-7: 2597701 | increased |
| Bacteroides fragilis YCH46: 817 | increased |
| Bacteroides caccae: 47678 | increased |
| Delftia tsuruhatensis: 180282 | increased |
| Ottowia sp. oral taxon 894: 1658672 | increased |
| Cardiobacterium hominis: 2718 | increased |
| Stenotrophomonas nitritireducens: 83617 | increased |
| Bacteroides heparinolyticus: 28113 | increased |
| Lysobacter oculi: 2698682 | increased |
| Acidovorax carolinensis: 553814 | increased |
| Acidovorax sp. T1: 1858609 | increased |
| Xanthomonas translucens pv. undulosa: 343 | increased |
| Melaminivora sp. SC2-9: 2109913 | increased |
| Aeromonas sp. ASNIH3: 1636608 | increased |
| Stenotrophomonas acidaminiphila: 128780 | increased |
| Ottowia oryzae: 2109914 | increased |
| Dermabacter jinjuensis: 1667168 | increased |
| Porphyromonas gingivalis W83: 837 | increased |
| Desulfomicrobium orale DSM 12838: 132132 | increased |
| Alicycliphilus denitrificans K601: 179636 | increased |
| Bacteroides caecimuris: 1796613 | increased |
| Ralstonia mannitolilytica: 105219 | increased |
| Desulfobulbus oralis: 1986146 | increased |
| Bacteroides xylanisolvens: 371601 | increased |
| Pseudomonas denitrificans (nom. rej.): 43306 | increased |
| Acidovorax ebreus TPSY: 721785 | increased |
| Bacteroides zoogleoformans: 28119 | increased |
| Acidovorax sp. JS42: 232721 | increased |
| Diaphorobacter sp. JS3050: 2735554 | increased |

### CONCLUSION

While the foregoing detailed description makes reference to specific exemplary embodiments, the present disclosure may be embodied in other specific forms without departing from its spirit or essential characteristics. Accordingly, the described embodiments are to be considered in all respects only as illustrative and not restrictive. For instance, various substitutions, alterations, and/or modifications of the inventive features described and/or illustrated herein, and additional applications of the principles described and/or illustrated herein, which would occur to one skilled in the relevant art and having possession of this disclosure, can be made to the described and/or illustrated embodiments without departing from the spirit and scope of the disclosure as defined by the appended claims. Such substitutions, alterations, and/or modifications are to be considered within the scope of this disclosure.

The scope of the invention is, therefore, indicated by the appended claims rather than by the foregoing description. The limitations recited in the claims are to be interpreted broadly based on the language employed in the claims and not limited to specific examples described in the foregoing detailed description, which examples are to be construed as non-exclusive and non-exhaustive. All changes which come within the meaning and range of equivalency of the claims are to be embraced within their scope.

It will also be appreciated that various features of certain embodiments can be compatible with, combined with, included in, and/or incorporated into other embodiments of the present disclosure. For instance, systems, methods, and/or products according to certain embodiments of the present disclosure may include, incorporate, or otherwise comprise features described in other embodiments disclosed and/or described herein. Thus, disclosure of certain features relative to a specific embodiment of the present disclosure should not be construed as limiting application or inclusion of said features to the specific embodiment.

In addition, unless a feature is described as being requiring in a particular embodiment, features described in the various embodiments can be optional and may not be included in other embodiments of the present disclosure. Moreover, unless a feature is described as requiring another feature in combination therewith, any feature herein may be combined with any other feature of a same or different embodiment disclosed herein. It will be appreciated that while features may be optional in certain embodiments, when features are included in such embodiments, they can be required to have a specific configuration as described in the present disclosure.

Likewise, any steps recited in any method or process described herein and/or recited in the claims can be executed in any suitable order and are not necessarily limited to the order described and/or recited, unless otherwise stated (explicitly or implicitly). Such steps can, however, also be required to be performed in a specific order or any suitable order in certain embodiments of the present disclosure.

Furthermore, various well-known aspects of illustrative systems, methods, products, and the like are not described herein in particular detail in order to avoid obscuring aspects of the example embodiments. Such aspects are, however, also contemplated herein.

1. A method for screening, detecting, and/or preventing renal/urinary disease in cats, the method comprising:
obtaining an oral microbial profile for a cat, the oral microbial profile comprising one or more microbial species present in an oral sample of the cat and a quantity or abundance of the one or more microbial species in the oral sample;
comparing the oral microbial profile to information in a database that identifies weighted correlations between:
(i) occurrence and/or prevalence of one or more renal/urinary diseases in cats; and
(ii) presence and/or abundance of various microbial species in oral microbiomes of cats, wherein the various microbial species comprise the one or more microbial species in the oral sample;
generating a risk score indicating a likelihood that the cat has the one or more renal/urinary diseases based on one or more matches between the oral microbial profile and the information in the database; and
categorizing the cat as developing the one or more renal/urinary diseases when the risk score meets or exceeds a predetermined threshold and, optionally, prescribing a therapeutic treatment protocol suitable for treating, mitigating, or preventing the development, advancement, or recurrence of the one or more renal/urinary diseases when the risk score meets or exceeds a predetermined threshold.

2. (canceled)

3. The method of claim 1, wherein obtaining the oral microbial profile for the cat comprises:
obtaining nucleic acid sequence data corresponding to microbial nucleic acid obtained from the oral sample;
analyzing the nucleic acid sequence data to identify the one or more microbial species present in the oral sample and quantifying the one or more microbial species; and
generating the oral microbial profile for the cat based on the identified and, optionally, quantified one or more microbial species.

4. The method of claim 3, wherein obtaining the microbial nucleic acid sequence data comprises:
sequencing microbial nucleic acid from the oral sample; and, optionally,
isolating the microbial nucleic acid from the oral sample.

5. The method of claim 4, wherein isolating the microbial nucleic acid from the oral sample comprises:
performing heat treatment on the oral sample; and
performing magnetic SPRI beads-based nucleic acid extraction on the heat-treated oral sample, with or without the addition of protein digesting reagents and detergents, to extract the microbial nucleic acid from the oral sample.

6. The method of claim 3, wherein analyzing the microbial nucleic acid sequence data comprises one or more of:
demultiplexing the nucleic acid sequence data;
trimming the nucleic acid sequence data;
mapping one or more unmapped reads onto a reference genome of the cat and/or onto existing microbial reference genomes;
classifying one or more reads as feline from the nucleic acid sequence data after mapping;
classifying one or more reads as microbial from the nucleic acid sequence data after mapping;
quantifying the one or more microbial reads;
transforming the quantified one or more microbial reads to account for sequence coverage biases using methods such as pairwise log ratio transformation; and
comparing compositional abundance patterns in the transformed one or more microbial reads against compositional abundance patterns in transformed data in a reference database comprising samples from cats that do not suffer from renal/urinary diseases, as well as samples from cats that suffer from specific renal/urinary diseases.

7. The method of claim 1, wherein comparing the oral microbial profile to the information in the database comprises one or more of:
calculating the abundance of the one or more microbial species in the oral sample;
identifying the one or more microbial species in the oral sample; and
comparing the abundance of the identified one or more microbial species in the oral sample to the presence and/or abundance of various microbial species in the oral microbiome of cats.

8. The method of claim 1, wherein generating the risk score comprises one or more of:
identifying one or more similarities between the compositional abundance of the one or more microbial species in the oral sample and the compositional abundance of various microbial species in the oral microbiome of cats contained in the database;
identifying one or more matches between the identity of the one or more microbial species in the oral sample and the presence of various microbial species in the oral microbiome of cats contained in the database;
quantifying the identified one or more similarities between the compositional abundance of the one or more microbial species in the oral sample and the compositional abundance of the one or more microbial species in the oral microbiome of cats contained in the database; and
identifying a presence of one or more predictive microbial species in the oral sample.

9. The method of claim 1, wherein the one or more renal/urinary diseases is selected from the group consisting of chronic kidney disease, cystine urinary crystals or stones, calcium oxalate urinary crystals or stones, struvite urinary crystals or stones, and idiopathic cystitis.

10. The method of claim 1 further comprising:
generating a report presenting (i) the risk score, (ii) an indication of developing the one or more renal/urinary diseases when the risk score meets or exceeds the predetermined threshold, (iii) a timing recommendation, (iv) optionally, one or more at home practices to improve renal/urinary health, (v) optionally, one or more diagnostic steps to diagnose the one or more renal/urinary diseases when the risk score meets or exceeds the predetermined threshold, and (vi) optionally, a prescription for the therapeutic treatment protocol; and, optionally,
communicating the generated report electronically to an owner of the cat and/or their veterinarian.

11. (canceled)

12. A computer system configured to indicate or predict renal/urinary disease in cats, the computer system comprising:
one or more processors; and
one or more computer-readable hardware storage devices having stored thereon instructions that are executable by the one or more processors to configure the computer system to:
receive microbial nucleic acid sequence data corresponding to microbial nucleic acid obtained from an oral sample taken from a cat;
analyze the microbial nucleic acid sequence data to identify one or more microbial species present in the oral sample and quantify the one or more microbial species;
generate an oral microbial profile for the cat based on the identified one or more microbial species and their respective abundances;
compare the oral microbial profile to information in a database that identifies weighted correlations between:
(i) occurrence and/or prevalence of one or more renal/urinary diseases in cats; and
(ii) presence and/or abundance of various microbial species in oral microbiomes of cats, wherein the various microbial species comprise the one or more microbial species in the oral sample;
identify one or more matches between the oral microbial profile and the information in the database;
generate a risk score indicating a likelihood that the cat has the one or more renal/urinary diseases based on the one or more matches between the oral microbial profile and the information in the database; and, optionally,
diagnose the cat as “developing” the one or more renal/urinary diseases when the risk score meets or exceeds a predetermined threshold,
prescribe a therapeutic treatment protocol suitable for treating or preventing the one or more renal/urinary diseases when the risk score meets or exceeds the predetermined threshold,
generate a report indicating (i) the risk score, (ii) an indication of developing the one or more renal/urinary diseases when the risk score meets or exceeds the predetermined threshold, (iii) a timing recommendation, (iv) optionally, one or more at home practices to improve renal/urinary health, (v) optionally, one or more diagnostic steps to diagnose the one or more renal/urinary diseases when the risk score meets or exceeds the predetermined threshold, and (vi) a prescription for the therapeutic treatment protocol, and/or
communicate the generated report electronically to an owner of the cat and/or their veterinarian.

13. The computer system of claim 12, wherein the instructions further configure the computer system to map one or more unmapped reads to a cat reference genome and/or map one or more reads to microbial reference genomes and, optionally, classify the reads as microbial or feline.

14. The computer system of claim 13, wherein the instructions further configure the computer system to identify at least one unmapped sequence read of the metagenomic sequence data and, optionally, classify the at least one unmapped read.

15. The computer system of claim 13, wherein feline oral microbiome samples having fewer than 10,000 classified microbial reads or more than 500,000 classified microbial reads are excluded from the comparison of the oral microbial profile for the cat against a database of defined microbial profiles.

16. The computer system of claim 12, wherein the instructions further configure the computer system to calculate an abundance of the one or more microbial species present in the oral sample.

17. The computer system of claim 16, wherein the abundance of the specific one or more microbial species present in the oral sample correlates to whether the specific one or more microbial species is a predictive microbial species for the specific renal/urinary disease.

18. The computer system of claim 16, wherein the instructions further configure the computer system to perform a pairwise log ratio comparison of the microbial abundance of the cat's oral sample against the information in the database.

19. The system of claim 18, wherein the specific one or more microbial species is a predictive microbial species when 50% or more of the maximum possible pairwise log ratio comparisons involving this microbe are significantly different when compared between a disease and a control cohort.

20.-42. (canceled)

43. A method for predicting the development of a renal/urinary, inflammatory, and/or endocrine disease in a cat, the method comprising:
obtaining an oral sample from a cat, the oral sample containing one or more microbial species;
isolating, from the oral sample, microbial nucleic acid of the one or more microbial species;
obtaining microbial nucleic acid sequence data corresponding to the microbial nucleic acid;
analyzing the microbial nucleic acid sequence data to identify one or more microbial species present in the oral sample and, optionally, quantifying the one or more microbial species;
generating an oral microbial profile for the cat based on the identified and, optionally, quantified one or more microbial species, the oral microbial profile comprising the one or more microbial species and, optionally, a quantity or relative abundance of the one or more microbial species in the oral sample;
comparing the oral microbial profile to information in a database that identifies weighted correlations between:
(i) occurrence and/or prevalence of one or more renal/urinary, inflammatory, and/or endocrine diseases in cats; and
(ii) presence and/or abundance of various microbial species in the oral microbiome of animals in the classification of the cat, wherein the various microbial species comprise the one or more microbial species in the oral sample;
generating a risk score indicating a likelihood of the cat developing the one or more renal/urinary, inflammatory, and/or endocrine diseases based on one or more matches between the oral microbial profile and the information in the database; and
indicating the cat as developing the one or more renal/urinary, inflammatory, and/or endocrine diseases when the risk score meets or exceeds a predetermined threshold.

44. The method of claim 43, wherein the oral microbial profile for the cat further comprises a percentage of gram-positive microbes and wherein the various microbial species in the oral microbiome of animals in the classification of the cat further comprises a percentage of gram-positive microbes.
